(19) United States
(12) Patent Application Publication
(10) Pub. No.: US 2020/0276338 A1
(43) Pub. Date: Sep. 3, 2020

(54) BIOREDUCTIVELY-ACTIVATED COMPOUNDS, THEIR PRODRUGS, RADIOPHARMACEUTICALS, THE COMPOSITIONS, AND THEIR APPLICATIONS IN MULTIMODAL THERANOSTIC MANAGEMENT OF HYPOXIA DISEASES INCLUDING CANCER

(71) Applicant: The Governors of the University of Alberta, Edmonton (CA)

(72) Inventors: Piyush KUMAR, Edmonton (CA); Hassan ELSAIDI, Edmonton (CA); Leonard Irving WIEBE, Edmonton (CA); Michael WEINFELD, Edmonton (CA); Carolynne Lacar RICARDO, Edmonton (CA)

(21) Appl. No.: 16/648,408

(22) PCT Filed: Sep. 19, 2018
(86) PCT No.: PCT/CA2018/051166

§ 371 Date: Mar. 18, 2020

Related U.S. Application Data
(60) Provisional application No. 62/560,512, filed on Sep. 19, 2017.

(51) Int. Cl. A61K 51/04; A61K 41/00; G01N 33/60; A61P 35/00

(52) U.S. Cl. CPC A61K 51/0497; A61K 51/0459; A61P 35/00; A61K 41/0038; G01N 33/60; A61K 51/0491

(57) ABSTRACT

Described herein are bioreductively-activated compounds, their prodrugs, radiopharmaceuticals, the compositions, and their application in multimodal theranostic management of hypoxia diseases including cancer.

### CROSS REFERENCE TO RELATED APPLICATION

This application claims priority to US patent application number U.S. 62/560,512, filed Sep. 9, 2017, the entire content of which is hereby incorporate by reference.

### FIELD

The present disclosure relates generally to bioreductively-activated compounds, their prodrugs, radiopharmaceuticals, the compositions, and their applications In multimodal theranostic management of hypoxia diseases including cancer.

### BACKGROUND

Solid tumors frequently exhibit rapid growth and aberrant vasculature, leading to oxygen (O₂) depletion (hypoxia) and poor nutrient supply.¹⁻⁸ Hypoxia alters cellular metabolism, which can trigger transcriptional responses, induce genetic alterations⁹⁻¹³ and activate the formation of transformed, self-renewing multipotent cancer stem cells (CSCs). Hypoxia promotes invasion, metastasis,^{14,15} tumor progression and recurrence.^{13,16-18} Hypoxic solid tumors are more resistant to radiotherapy and (due to impaired drug delivery)⁶ to chemotherapy.^{4,15, 19-21} Tumor hypoxia thus poses a formidable challenge to therapeutic interventions and leads to poor local control and overall survival.^{22,23}

### SUMMARY

In one aspect there is described a compound of formula (I), or any prodrug, pharmaceutically acceptable salt, metabolite, polymorph, solvate, hydrate, stereoisomer, radioisotope or tautomer thereof

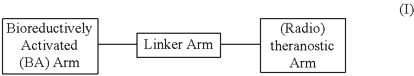

wherein BA comprises one or more of 2/4/5-substituted nitroimidazoles, substituted benzotriazene-1,4-dioxides, substituted 1,2,3/1,2,4-triazoles, substituted 1,4-benzoquinones, of combination of two homo- or hetero BA moieties,

wherein Linker Arm is —C₁₋₁₆ alkane, alkene, alkyne, alicyclic, aromatic with or without hetero atoms as in ethers, amines, esters, acids, amides; 5 and 6 membered rings with the substitutions as described above, both monosaccharides and diasaccharides,

wherein the (Radio)theranostic arm comprises ^{18/19}F, ^{123/124/125/127/131}I, Lu-177, Ga-68, ^{99m}Tc, Gd, etc.

In one aspect there is described a compound of formula (II), or any prodrug, pharmaceutically acceptable salt, metabolite, polymorph, solvate, hydrate, stereoisomer, radioisotope or tautomer thereof,

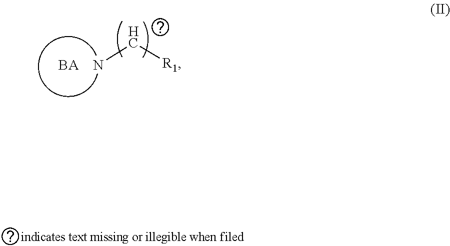

wherein

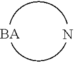

(BA) is a bioreductively-activated molecule, for example, 2/4/5-nitroimidazoles (such as in F-MISO), or substituted with cyclic moieties, or sugar substituted moieties (both pentoses as in FAZA [substituted or unsubstituted] and IAZA [substituted or unsubstituted], and hexoses, disaccharides and trisaccharides in all configurations; for example, as in glucoses, galactoses, fructoses, other substituted moieties nitroimidazoles, benzotriazene-1,4-dioxides e.g. tirapazamine, and analogs thereof, substituted 1,2,4-triazoles, substituted tetrahydroisoquinolines, substitutes benzoquinones, e.g. AQ4N;

wherein R₁ is unsubstituted, or substituted molecule with one or more —OH groups, wherein the one or more —OH group is substituted with an alkyl, aralkyl ether, ester, amine or a thiol, and the remaining free —OH group is replaced by a radiohalogen, H, halogen, azide, amine-substituted/unsubstituted, —OH, substituted —OH, —OSO₂R₃;

wherein R₃ is alkyl sulfonyl (such as methanesulfonyl, or arylsulfonyl e.g. tosyl, nosyl, trifly)-substituted alkan/alkene/alkyne/alkoxy/alkoxyalkenyl and alkoxyalkynyl chains;

wherein n is C₁-C₂₂.

In one example, the sugar containing a bioreductively activated molecule is substituted with an ether or ester moiety at 2′ and/or 3′ positions, and a halogen/pseudohalogen (F/I/OTosyl/ONosyl/OTriflyl/OMesyl) substituted at 2′- or 3′- or 5′-OH of a sugar with or without a linker.

In one example, said Acyclic or cyclic substituents linked to the BA moieties are further substituted with R₁, where R₁=alkane/alkene/alkyne/alkoxy/alkoxyalkyl/alkoxyalkenyl and alkoxyalkynyl chains (C1-C22), where R₂═H, halogens, Azide, —OH, substituted —OH, —OSO₂R₃(R₃=alkyl sulfonyl e.g., methanesulfonyl, or arylsulfonyl e.g., tosyl, nosyl, triflyl).

In one example, said bioreductively activated molecule is an azomycin-based compound, such as retinoyl IAZA [Ret-IAZA], retinoyl FAZA [Ret-FAZA], but are not limited to sugar conjugated family; in benzotriazene-1,4-dioxide based molecules include tirapazamine (TPZ)-based compounds, for example (C2/C4/C6 gluc substituted-TPZ), and all related precursors to synthesize the corresponding halogenated (F, Cl, Br, I, At) derivatives.

In one example, said

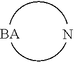

is

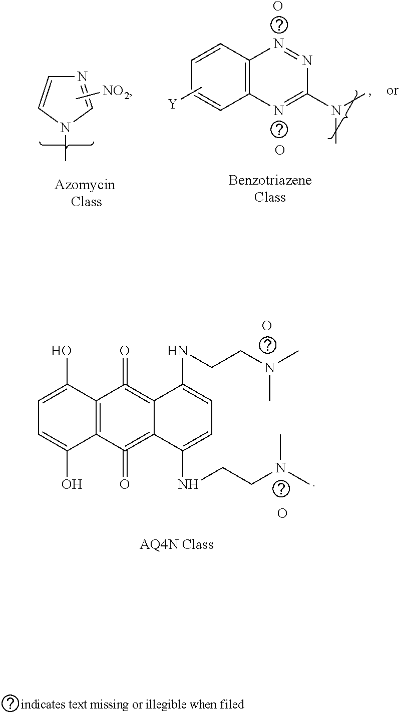

In one example, said benzotriazene class is

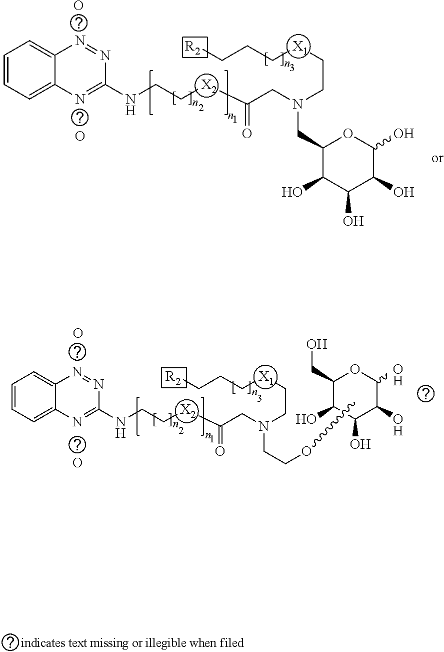

wherein R₂ is I, F, Br, Cl, At, N₃;

wherein X₁ is C, N, O, S;

wherein X₂ is C, N, O, S

wherein n₁ is 1-22;

wherein n₂ is 1-22;

wherein n₃ is 1-22.

In one aspect there is described a compound of formula (II), or any prodrug, pharmaceutically acceptable salt, metabolite, polymorph, solvate, hydrate, stereoisomer, radioisotope or tautomer thereof,

Y-L-BA (11)

wherein BA is a bioreductively-activated molecule, for example, 2/4/5-nitroimidazoles (such as in F-MISO), or substituted with cyclic moieties, or sugar substituted moieties (both pentoses as in FAZA [substituted or unsubstituted] and IAZA [substituted or unsubstituted], and hexoses, disaccharides and trisaccharides in all configurations; for example, as in glucoses, galactoses, fructoses, other substituted moieties nitroimidazoles, benzotriazene-1,4-dioxides e.g. tirapazamine, and analogs thereof, substituted 1,2,4-triazoles, substituted tetrahydroisoquinolines, substitutes benzoquinones, e.g. AQ4N;

wherein L is a linker, such as cyclic or acyclic moiety with up to C8 chain, which can be further substituted by alkane/alkene/alkyne/alkoxy/alkoxyalkyl/alkoxyalkenyl or alkoxyalkynyl chains (C1-C22) containing H, halogen, azide, —OH, substituted —OH, —OSO₂R₃(R₃ is alkyl sulfonyl e.g., methanesulfonyl or arylsulfonyl e.g. tosyl, nosyl, triflyl), for example C1-α/β-substituted arabinofuranoses/pentoses/hexoses (e.g., glucose, disaccharide etc.) where the other —OH groups except one in the sugar ring are either unsubstituted, or substituted with alkyl aralkyl ethers, esters, amines or thiols; remaining free —OH group is replaced by radio halogen,

wherein Y is a ligand (e.g., tetradentate ligand for example DOTA or NOTA or PnAO.

In one aspect there is described a radio labeled compound comprising a compound of any one of claims 1 to 8, wherein said radio label is a radioisotope, a radiohalogen, F-18, I-123/124/125/131, F-18 labelled dipivaloyl 5′-¹⁸FAZA and I-123/124/125/131-labelled diretinoyl-^{123/124/125/131}IAZA, radiolabeled ret-IAZA or retinoyl FAZA, for both α- and β-conformers.

In one aspect there is described a pharmaceutical comprising a compound of any one of claims 1 to 9, or a radio labeled compound of claim 8, and one or more inert carriers and/or diluents.

In one aspect there is described a use of a compound of any one of claims 1 to 8, a radio labeled compound of claim 9, or a pharmaceutical composition of claim 10, as a diagnostic agent in a subject.

In one aspect there is described a use of a compound of any one of claims 1 to 8, a radio labeled compound of claim 9, or a pharmaceutical composition of claim 10, as a therapeutic agent in a subject.

In one aspect there is described a use of a compound of any one of claims 1 to 8, a radio labeled compound of claim 9, or a pharmaceutical composition of claim 10, as a diagnostic and therapeutic agent in a subject.

In one aspect there is described a use of a compound of any one of claims 1 to 8, a radio labeled compound of claim 9, or a pharmaceutical composition of claim 10, as an imaging agent in a subject.

In one aspect there is described a use of a compound of any one of claims 1 to 8, a radio labeled compound of claim 9, or a pharmaceutical composition of claim 10, as a radiosensitization agent in a subject.

In one aspect there is described a use of a compound of any one of claims 1 to 8, a radio labeled compound of claim 9, or a pharmaceutical composition of claim 10, as a chemosensitization agent in a subject.

In one aspect there is described a use of a compound of any one of claims 1 to 8, a radio labeled compound of claim 9, or a pharmaceutical composition of claim 10 in the treatment of a hypoxia tumours and/or cancers, diabetes, inflammatory arthritis, anaerobic bacterial infection, stroke, brain trauma or transplant rejection.

In one example, said subject is a human.

### BRIEF DESCRIPTION OF THE FIGURES

Embodiments of the present disclosure will now be described, by way of example only, with reference to the attached Figures.

FIG. 1 is a graph depicting TLC of purified 2′-O-Retinoyl [¹³¹I]IAZA.

FIG. 2 is a graph depicting radiochromatogram of pure Acetyl [¹³¹I]l-GAZ

FIG. 3 is a graph depicting radio-TLC of purified [¹³¹I]l-TPZ after passing through alumina cartridge.

FIG. 4 is a graph depicting a radiochromatogram of [¹³¹I]IG-6-TPZ after alumina cartridge facilitated solid phase purification.

FIG. 5 depicts MTT Assay for PK-CR-IA in FaDu Cells.

FIG. 6 depicts MTT Assay for IAZA in FaDu Cells.

FIG. 7 depicts MTT assay for retinoic acid in FaDu cells.

FIG. 8 depicts Cytotoxicity of HE-1-57-B23 in FaDu cells.

FIG. 9 depicts Cytotoxicity of HE-1-57-B23 in U251 Cells

FIG. 10 depicts Cytotoxicity of TPZ-OH in FaDu cells

FIG. 11 depicts Cytotoxicity of TPZ-OH in U251 cells.

FIG. 12 depicts Cytotoxicity of TPZ-OH in PC3 cells.

FIG. 13 depicts Cytotoxicity of HE-B-104 in FaDu cells.

FIG. 14 depicts Cytotoxicity of HE-B-104 in U251 cells.

FIG. 15 depicts Cytotoxicity of HE-B-104 in PC3 cells.

FIG. 16 depicts Cytotoxicity of Azido-TPZ in FaDu cells

FIG. 17 depicts Cytotoxicity of Azido-TPZ in U251 cells.

FIG. 18 depicts Cytotoxicity of Azido-TPZ in PC3 cells.

FIG. 19 depicts Cytotoxicity of HE-1-127-B48 in FaDu cells

FIG. 20 depicts Radiosensitization of FaDu cells by PK-CR-IA-CFA assay at 0-14 Gray.

FIG. 21 depicts Radiosensitization of FaDu cells by HE-1-57-B23.

FIG. 22 depicts Radiosensitization of U-251 cells by HE-1-57-B23.

FIG. 23 depicts Radiosensitization of PC-3 cells by HE-1-57-B23.

FIG. 24 depicts Radiosensitization of PC3 cells by HE-1-127-B48.

FIG. 25 depicts Radiosensitization of U251 cells by HE-1-127-B48.

FIG. 26 depicts Radiosensitization of U251 cells by TPZ.

FIG. 27 depicts. Radiosensitization of FaDu cells by TPZ.

FIG. 28 depicts Histological sections of FaDu tumors grown in mice, representing No treatment (A) and Radiation (10 Gy) alone treatment (B).

FIG. 29 depicts Histological sections of FaDu tumors grown in mice, representing IAZA treatment (A) and IAZA plus Radiation (10 Gy) treatment (B).

### DETAILED DESCRIPTION

Described herein is the development of hypoxia-targeted bioreductively-activated molecules that demonstrate multi-fold theranostic (therapeutic+diagnostic) potential for the management of oxygen-deficient, therapy-resistant tumors that are found in many kinds of cancers.

Using a ‘single molecule’ approach these molecules can bestow molecular imaging of hypoxic cells, as well as provide chemotherapeutic effects, molecular radiotherapy (MRT) effects when labelled with a therapeutic radioisotope, and also radiosensitization therapy in conjunction with conventional radiotherapy.

Thus, in one example, described herein is an effective multimodal theranosis of hypoxic tumors. In some examples, the compounds and compositions herein may be useful in management of several other diseases that demonstrate physiological hypoxia, including diabetes, inflammatory arthritis, anaerobic bacterial infections, stroke, brain trauma and transplant rejection.

Unless defined otherwise, all technical and scientific terms used herein have the same meaning as commonly understood by one of ordinary skill in the art to which this disclosure belongs.

As used herein, the term “hydrocarbon,” used alone or in combination, refers to a linear, branched or cyclic organic moiety comprising carbon and hydrogen, for example, alkyl, alkene, alkyne, and aryl, which may each be optionally substituted. In some examples, a hydrocarbon may, for example, comprise about 1 to about 60 carbons, about 1 to about 40 carbons, about 1 to about 30 carbons, about 1 to about 20 carbons, about 1 to about 10 carbons, about 1 to about 9 carbons, about 1 to about 8 carbons, about 1 to about 6 carbons, about 1 to about 4 carbons, or about 1 to about 3 carbons. In some embodiments, hydrocarbon comprises 10 carbons, 9 carbons, 8 carbons, 7 carbons, 6 carbons, 5 carbons, 4 carbons, 3 carbons, 2 carbons, or 1 carbon.

As used herein, the term “alkyl” refers to straight or branched hydrocarbon. An alkyl may be linear, branched, cyclic, or a combination thereof, and may contain, for example, from one to sixty carbon atoms. Examples of alkyl groups include but are not limited to ethyl, ethyl, propyl, isopropyl, cyclopropyl, butyl isomers (e.g. n-butyl, iso-butyl, tert-butyl, etc.) cyclobutyl isomers (e.g. cyclobutyl, methylcyclopropyl, etc.), pentyl isomers, cyclopentane isomers, hexyl isomers, cyclohexane isomers, and the like.

As used herein, the term “linear alkyl” refers to a chain of carbon and hydrogen atoms (e.g., ethane, propane, butane, pentane, hexane, etc.). A linear alkyl group may be referred to by the designation —(CH₂)_{q}CH₃, where q is, for example, 0-59. The designation “C₁₋₁₂ alkyl” or a similar designation, refers to alkyl having from 1 to 12 carbon atoms such as methyl, ethyl, propyl isomers (e.g. n-propyl, isopropyl, etc.), butyl isomers, cyclobutyl isomers (e.g. cyclobutyl, methylcyclopropyl, etc.), pentyl isomers, cyclopentyl isomers, hexyl isomers, cyclohexyl isomer, heptyl isomers, cycloheptyl isomers, octyl isomers, cyclooctyl isomers, nonyl isomers, cyclononyl isomers, decyl isomer, cyclodecyl isomers, etc. Similar designations refer to alkyl with a number of carbon atoms in a different range.

As used herein, the term “branched alkyl” refers to a chain of carbon and hydrogen atoms, without double or triple bonds that contains a fork, branch, and/or split in the chain. “Branching” refers to the divergence of a carbon chain, whereas “substitution” refers to the presence of non-carbon/non-hydrogen atoms in a moiety.

As used herein, the term “cycloalkyl” refers to a completely saturated mono- or multi-cyclic hydrocarbon ring system. When composed of two or more rings, the rings may be joined together in a fused, bridged or spiro-connected fashion. A cycloalkyl group may be unsubstituted, substituted, branched, and/or unbranched. Typical cycloalkyl groups include, but are not limited to, cyclopropyl, cyclobutyl, cyclopentyl, cyclohexyl, and the like. If substituted, the substituent(s) may be an alkyl or selected from those indicated above with regard to substitution of an alkyl group unless otherwise indicated. Unless specified otherwise (e.g., substituted cycloalkyl group, heterocyclyl, cycloalkoxy group, halocycloalkyl, cycloalkylamine, thiocycloalkyl, etc.), an alkyl group contains carbon and hydrogen atoms only.

As used herein, the term “heteroalkyl” refers to an alkyl group, wherein one or more carbon atoms are independently replaced by one or more heteroatoms (e.g., oxygen, sulfur, nitrogen, phosphorus, silicon, or combinations thereof). The alkyl group containing the non-carbon substitution(s) may be a linear alkyl, branched alkyl, cycloalkyl (e.g., cycloheteroalkyl), or combinations thereof. Non-carbons may be at terminal locations (e.g., 2-hexanol) or integral to an alkyl group (e.g., diethyl ether).

The term “alkoxy”, used alone or in combination, means the group —O— alkyl.

The term “alkenyl”, used alone or in combination, means a straight or branched chain hydrocarbon having at least 2 carbon atoms, which contains at least one carbon-carbon double bond.

The term “haloalkyl” refers to an alkyl in which one or more hydrogen has been replaced with same or different halogen.

The term “alkynyl”, used alone or in combination, means a straight or branched chain hydrocarbon having at least 2 carbon atoms, which contains at least one carbon-carbon triple bond

The term “alkoxyalkyl” means a moiety of the formula —R′-R″, where R′ is alkylene and R″ is alkoxy.

The term “aryl”, used alone or in combination, means an aromatic carbocyclic moiety of up to 60 carbon atoms, which may be a single ring (monocyclic) or multiple rings fused together (e.g., bicyclic or tricyclic fused ring systems).

The term “alkylene” refers to divalent aliphatic hydrocarbyl groups preferably having from 1 to 6 and more preferably 1 to 3 carbon atoms that are either straight-chained or branched.

The terms “amine” or “amino” as used herein are represented by a formula NA1A2A3, where A1, A2, and A3 can be, independently, hydrogen or optionally substituted alkyl, cycloalkyl, alkenyl, cycloalkenyl, alkynyl, cycloalkynyl, aryl, or heteroaryl group as described herein. In specific embodiments amine refers to any of NH2, NH(alkyl), NH(aryl), N(alkyl)2, N(alkyl)(aryl), and N(aryl)2.

As used herein, the term “substituted” means that the referenced group (e.g., alkyl, aryl, etc.) comprises a substituent group. The term “optionally substituted”, as used herein, means that the referenced group (e.g., alkyl, cycloalkyl, etc.) may or may not be substituted with one or more additional group(s).

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R_{0.5}H₂O)), and polyhydrates (x is a number greater than 1, e.g., dihydrates (R.₂H₂O) and hexahydrates (R.6H₂O)).

The term “tautomers” refer to compounds that are interchangeable forms of a particular compound structure, and that vary in the displacement of hydrogen atoms and electrons. Thus, two structures may be in equilibrium through the movement of it electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base. Tautomeric forms may be relevant to the attainment of the optimal chemical reactivity and biological activity of a compound of interest.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorphs” refers to a crystalline form of a compound (or a salt, hydrate, or solvate thereof) in a particular crystal packing arrangement. All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refer to compounds, including derivatives of the compounds described herein, which have cleavable groups and become by solvolysis or under physiological conditions the compounds described herein, which are pharmaceutically active in vivo.

As used herein, “derivative” refers to any compound having the same or a similar core structure to the compound but having at least one structural difference, including substituting, deleting, and/or adding one or more atoms or functional groups. The term “derivative” does not mean that the derivative is synthesized from the parent compound either as a starting material or intermediate, although this may be the case.

The term “metabolite” includes any compound into which a compound as described here can be converted in vivo once administered to the subject.

The term “subject”, may refer to an animal, and can include, for example, domesticated animals, such as cats, dogs, etc., livestock (e.g., cattle, horses, pigs, sheep, goats, etc.), laboratory animals (e.g., mouse, rabbit, rat, guinea pig, etc.), mammals, non-human mammals, primates, non-human primates, rodents, birds, reptiles, amphibians, fish, and any other animal. In a specific example, the subject is a human.

The terms “administer,” “administering,” or “administration” refers to implanting, absorbing, ingesting, injecting, inhaling, or otherwise introducing an inventive compound, or a pharmaceutical composition thereof, in or on a subject.

The terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein, such as a fungal or protozoan infection. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

A “therapeutically effective amount” of a compound or composition described herein is an amount sufficient to provide a therapeutic benefit in the treatment of a condition or to delay or minimize one or more symptoms associated with the condition. A therapeutically effective amount of a compound or composition means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

A “prophylactically effective amount” of a compound or composition described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

As used herein, the term “pharmaceutical composition” refers to the combination of an active agent with a carrier, inert or active, making the composition suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants.

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

The term “sample” or “biological sample” refers to any sample including tissue samples (such as tissue sections and needle biopsies of a tissue); cell samples (e.g., cytological smears or samples of cells obtained by microdissection); samples of whole organisms; or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucus, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. Biological samples also include those biological samples that are transgenic, such as transgenic oocyte, sperm cell, blastocyst, embryo, fetus, donor cell, or cell nucleus.

The term “radiosensitizer”, as used herein, refers to a compound or composition which when administered to a subject in therapeutically effective amounts to increases the sensitivity of the cells to ionizing radiation and/or to promote the treatment of diseases which are treatable with ionizing radiation.

In some examples, non-limiting examples of radiation therapy include external beam radiation therapy (EBRT or XRT), tele therapy, brachytherapy, sealed source radiation therapy, systemic radioisotope therapy (SRT), molecular radiotherapy (MRT), endoradiotherapy, unsealed source radiation therapy, intraoperative radiation therapy (IORT), targeted intraoperative radiation therapy (TARGIT), intensity-modulated radiation therapy (IMRT), volumetric modulated arc therapy (VMAT), particle therapy, and auger therapy.

The term “chemosensitizer”, as used herein, refers to a compound of composition which when administered to a subject in therapeutically effective amounts to increase the sensitivity of cells to chemotherapy and/or promote the treatment of diseases which are treatable with chemo therapeutics.

The term “fluorescent dye” as used herein refers to moieties that absorb light energy at a defined excitation wavelength and emit light energy at a different wavelength.

In one example, the term “radiochemical” as used herein refers to an organic, inorganic or organometallic compound comprising a covalently-attached or coordinately-attached (ligand) radioactive isotope, inorganic radioactive ionic solution, or radioactive gas, particularly including radioactive molecular imaging probes intended for administration to a patient (e.g., by inhalation, ingestion or intravenous injection) for tissue imaging purposes, which are also referred to in the art as radiopharmaceuticals, radiotracers or radioligands.

The term “radioactive isotope” or “radioactive element” refers to isotopes exhibiting radioactive decay (for example, emitting positrons, beta particles, gamma radiations etc.) and radiolabeling agents comprising a radioactive isotope.

Isotopes or elements are also referred to in the art as radioisotopes or radionuclides.

Radioactive isotopes are named herein using various commonly used combinations of the name or symbol of the element and its mass number (e.g., ¹⁸F, F-18, or fluorine-18). Non limiting examples of radioactive isotopes include 1-124, F-18 fluoride, C-I 1, N-13, and 0-15, I-123, I-124, I-127, I-131, Br-76, Cu-64, Tc-99m, Y-90, Ga-67, Cr-51, Ir-192, Mo-99, Sm-153 and Tl-201. Other examples of radioactive isotopes include: As-72, As-74, Br-75, Co-55, Cu-61, Cu-67, Ga-68, Ge-68, 1-125, 1-132, In-111, Mη-52, Pb-203 and Ru-97.

As used herein, the term “theranostic” refers to a combination of a specific therapy and diagnostic.

As used herein in connection with a measured quantity, the term “about” refers to the normal variation in that measured quantity that would be expected by the skilled artisan making the measurement and exercising a level of care commensurate with the objective of the measurement and the precision of the measuring equipment used. Unless otherwise indicated, “about” refers to a variation of +/−10% of the value provided.

The general structure of the compounds described (Scheme. 1 and Scheme. 2) include a bioreductively-activated (BA) moiety-derived acyclic molecules, e.g., 2/4/5-nitroimidazoles (as in F-MISO), or substituted with cyclic moieties, or sugar substituted moieties (both pentoses as in FAZA [substituted or unsubstituted] and IAZA [substituted or unsubstituted], and hexoses, disaccharides and trisaccharides in ALL configurations; for example, as in glucoses, galactoses, fructoses, other substituted moieties). Examples of other BA arms claimed under the invention include substituted or unsubstituted benzo-1,2,4-triazene-1,4-dioxides (e.g., substituted tirapazamines); substituted benzoquinones e.g., as in AQ4N, substituted triazoles as in HX4, their precursors, and their derivatives.

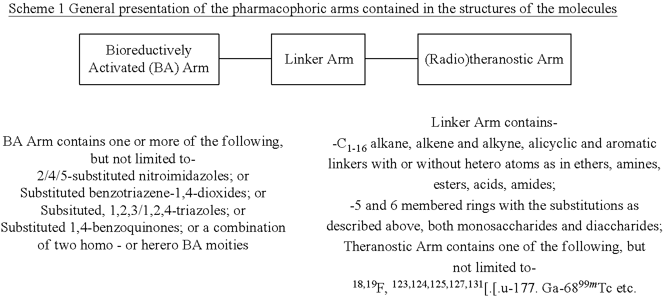

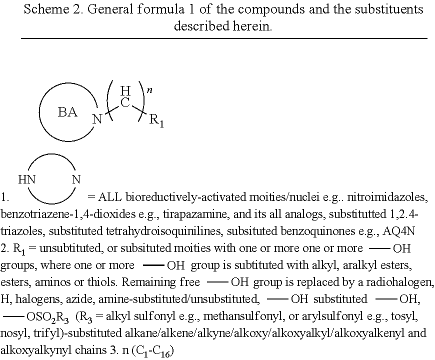

Sugar containing bioreductively activated molecules described above may further be substituted with an ether or ester moiety at 2′ and/or 3′ and/or 5′ positions, and a halogen/pseudohalogen (F/I/OTosyl/ONosyl/OTriflyl/OMesyl) substituted at 2′-, or 3′ or 5′-OH of a sugar with or without a linker (Scheme 2);

Acyclic or cyclic substituents linked to the BA moieties are further substituted with R₁, where R₁=alkane/alkene/alkyne/alkoxy/alkoxyalkyl/alkoxyalkenyl and alkoxyalkynyl chains (C₁-C₂₂), where R₂═H, halogens, Azide, —OH, substituted —OH, —OSO₂R₃(R₃=alkyl sulfonyl e.g., methanesulfonyl, or arylsulfonyl e.g., tosyl, nosyl, triflyl);

Examples of bioreductively activated molecules in azomycin-based compounds with sugar include retinoyl IAZA [Ret-IAZA], retinoyl FAZA [Ret-FAZA], but are not limited to sugar conjugated family; in benzotriazene-1,4-dioxide based molecules include tirapazamine (TPZ)-based compounds, for example (C2/C4/C6 glucose substituted-TPZ), and ALL related precursors to synthesize the corresponding halogenated (F, Cl, Br, I, At) and functionalized (including N₃, fluorescent moieties) derivatives. Claims on bioreductively activated molecules is however not limited to these classes.

Non-limiting examples the classes of bioreductively activated cores are described in Scheme 3

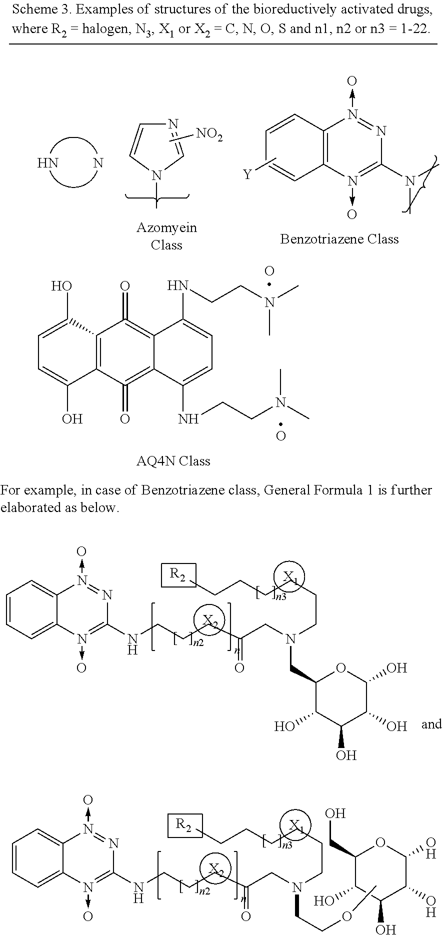

Embodiments from the classes of the BA drugs synthesized covered by the general formula 1 (Scheme. 3) are provided below.

### Class 1: 2′,3′-Di-O-Substituted Esters of 5′-Halo α/β-AZA

Three methods have been developed to synthesize this class of compounds.

Method A:

Into an oven vacuum-dried round bottom flask equipped with magnetic stir bar, azomycin-based sugar, for example FAZA (1 eq), was dissolved in anhydrous pyridine (3 mL). After which, the desired acid chloride (4 eq) was added dropwise to this solution and the resulting mixture was stirred under Argon at room temperature for a period of 24 h. Crude reaction mixture was concentrated in vacuo and passed through a silica gel column using 8:2 (v/v) hexane-ethyl acetate as eluent to give the desired product.

### Example 1

Synthesis of 5′-Fluoro-2′,3′-di-O-pivaloylarabinofuranosyl-2-nitroimidazole (Dipivaloyl FAZA, Compound 1) as a representative of the Class 1 compounds synthesized via Method A is described below. Following Method A, FAZA (0.08 g, 0.32 mmol, 1.0 eq) was dissolved in anhydrous pyridine and reacted with pivaloyl chloride (159 μL, 1.29 mmol, 4 eq) under Argon at 22° C. for a period of 24 h. Crude reaction mixture was concentrated in vacuo and passed through a silica gel column using 8:2 (v/v) hexane-ethyl acetate as eluent to give 0.1242 g (yield—0.299 mmol; 93%) of compound 1 as a white solid. ¹H NMR (400 MHz, CDCl₃) δ 7.35 (d, J=1.2 Hz, 1H, imidazole, H-5), 7.23 (d, J=1.1 Hz, 1H, imidazole, H-4), 6.68 (d, J=0.9 Hz, 1H, H-1′), 5.35 (d, J=1.0 Hz, 1H, H-2′), 5.07 (dd, J=2.0, 1.0 Hz, 1H, H-3′), 4.74 (dd, J=4.1, 1.7 Hz, 1H, H-4′), 4.69-4.58 (m, 2H, H-5′), 4.58 (dd, J=5.0, 2.3 Hz, 1H), 1.28 (s, 9H, 3×CH₃), 1.10 (s, 9H, 3×CH₃) ppm; 13C NMR (125 MHz, CDCl₃) δ 177.16 (C═O), 176.41 (C═O), 144.09 (imidazole, C-2), 128.55 (imidazole, C-4), 121.76 (imidazole, C-5), 93.59 (C-1′), 86.94 (C-5′), 81.51 (C-2′), 80.97 (C-4′), 76.19 (C-3′), 38.78 (pivaloyl, C), 38.57 (pivaloyl C), 26.83 (CH₃), 26.79 (CH₃) ppm; ¹⁹F NMR (376 MHz, Chloroform-d) δ −228.74 (td, JF-H-5′=46.6, JF-H-4′=22.9 Hz) ppm; HR-MS (ESI): m/z: 438.1652 [M+Na]+.

### Example 2: Synthesis of 5′-Iodo-2′,3′-dipivaloylarabinofuranosyl-2-nitroimidazole (Dipivaloyl IAZA, Compound 1a)

Yield 55 mg.

Method B.

Examples of novel compounds described under Class 1 synthesized by this methodology include: 5′-lodo-2′,3′-di-O-retinoyl arabinofuranosyl-2-nitroimidazole (Diretinoyl IAZA, Compound 3 in 71% yield, ¹H NMR, ¹³C NMR, HR-MS); 5′-Fluoro-2′,3′-diretinoylarabinofuranosyl-2-nitroimidazole (Diretinoyl FAZA, Compound 2 in 40% yield, ¹H NMR. ¹³C NMR, HR-MS); 5′-O-tosyl-2′,3′-diretinoylarabinofuranosyl-2-nitroimidazole (Diretinoyl AZA tosylate, Compound 4; 18% yield, ¹H NMR, ¹³C NMR, HR-MS); 2′-O-retinoyl FAZA (Monoretinoyl FAZA or Compound 5; 59% yield, ¹H NMR, 13C NMR, HR-MS).

### Synthesis of Diretinoyl IAZA (3) is an Example of Synthesizing the Compounds Categorized Under Class 1 Using Method B

Retinoyl chloride was prepared by adding oxalyl chloride (0.058 g, 40 μL, 0.451 mmol) dropwise into a solution of retinoic acid (0.0896 g, 0.2961 mmol) in 6 mL dry toluene and the solution was stirred at room temperature for 1 h under Argon. After which, toluene was evaporated carefully under reduced pressure and the residue was dissolved in 2 mL dry toluene and added to a solution containing IAZA (0.050 g, 0.141 mmol) and dimethylaminopyridine (DMAP) (0.0546 g, 0.447 mmol) in 5 mL dry toluene at 0° C. The reaction was allowed to proceed for one hour at 0° C. and then heated to reflux for 3 h. Crude mixture was concentrated in vacuo and purified via column chromatography using 9.5:0.5 (v/v) chloroform-ethyl acetate to furnish 3 as a yellow solid (0.092 g, 0.1 mmol) in 71% yield. ¹H NMR (500 MHz, CDCl₃) δ 7.44 (d, J=1.2 Hz, 1H, imidazole H-5), 7.24 (d, J=1.3 Hz, 1H, imidazole H-4), 7.10 (ddd, J=18.8, 15.0, 11.4 Hz, 2H, retinoyl H-5 and H-5′), 6.76 (s, 1H, H-1′), 6.35 (d, J=15.4 Hz, 3H, retinoyl H-4, H-6 and H-6′), 6.27 (d, J=15.0 Hz, 1H, retinoyl H-4′), 6.23-6.14 (m, 4H, retinoyl H-8, H-8′, H-9 and H-9′), 5.86 (s, 1H, retinoyl H-2), 5.56 (s, 1H, retinoyl H-2′), 5.53 (s, 1H, H-2′), 5.24 (s, 1H, H-3′), 4.74 (ddd, J=7.9, 5.6, 1.9 Hz, 1H, H-4′), 3.58 (dd, J5′-4′=10.7, Jgem=5.7 Hz, 1H, H-5′), 3.48 (dd, J5′-4′=10.6, Jgem=8.1 Hz, 1H, H-5′), 2.40 (s, 3H, retinoyl C-3 CH₃),), 2.36 (s, 3H, retinoyl C-3 CH3), 2.07-2.02 (m, 4H, cyclohexene H-3, H-3′), 2.05 (s, 3H, retinoyl C-7 CH₃), 2.04 (s, 3H, retinoyl C-7′ CH₃), 1.75 (s, 3H, cyclohexene C-2 CH₃), 1.74 (s, 3H, cyclohexene C-2′ CH3), 1.65 (p, J=6.0 Hz, 4H, cyclohexene H-4 and H-4′), 1.53-1.47 (m, 4H, cyclohexene H-5 and H-5′), 1.07 (s, 6H, cyclohexene C-6 2×CH₃) 1.06 (s, 6H, cyclohexene C-6′ 2×CH₃) ppm; ¹³C NMR (125 MHz, CDCl₃) δ 164.94 (retinoyl C-1, C═O), 164.31 (retinoyl C-1′, C═O), 156.88 (retinoyl C-3), 156.53 (retinoyl C-3′), 144.07 (imidazole C-2), 140.98 (retinoyl, C-7), 140.92 (retinoyl, C-7′), 137.66 (cyclohexene C-1), 137.63 (cyclohexene C-1′), 137.13 (retinoyl C-8), 137.08 (retinoyl C-8′), 134.30 (retinoyl C-4), 134.18 (retinoyl C-4′), 132.71 (retinoyl C-5 and C-5′), 130.35 (cyclohexene C-2), 130.29 (cyclohexene C-2′), 129.48 (retinoyl C-6), 129.40 (retinoyl C-6′), 129.29 (retinoyl C-9), 129.20 (retinoyl C-9′), 128.34 (imidazole C-4), 122.46 (imidazole C-5), 115.47 (retinoyl C-2 and C-2′), 93.38 (C-1′), 88.06 (C-2′), 80.82 (C-3′), 77.53 (C-4′), 39.62 (cyclohexene C-5 and C-5′), 34.28 (cyclohexene C-6), 33.14 (cyclohexene C-3), 29.70 (cyclohexene C-6 CH3), 28.98 (cyclohexene C-6′ CH3), 21.77 (cyclohexene C-2 CH3), 21.76 (cyclohexene C-2′ CH3), 19.22 (cyclohexene C-4 and C-4′), 14.27 (retinoyl C-3 CH3), 14.15 (retinoyl C-3′ CH3), 13.00 (retinoyl C-7 CH3), 12.99 (retinoyl C-7′ CH₃) ppm; HR-MS (ESI): m/z: 942.3539 [M+Na]+).

Method C:

General Method:

Azomycin nucleoside (1 eq), retinoic acid (2.1 eq), and DMAP (2.1 eq) were dissolved in anhydrous CH₂Cl₂ under Argon. In a separate round bottom flask, dicylohexyl carbodiimide (DCC, 2.1 eq) was dissolved in anhydrous CH2Cl2 and then added into the solution of sugar and retinoic acid with stirring. The resulting mixture was stirred at room temperature overnight in the dark. After which, the mixture was filtered, concentrated in vacuo and purified via column chromatography using 9.5:0.5 (v/v) CH₂Cl₂-methanol as eluent.

Examples of compounds synthesized using this methodology include: Diretinoyl IAZA (Compound 3; 60% yield, ¹H NMR, ¹³C NMR, HR-MS); 5′-Diretinoyl FAZA (Compound 2, 87% yield, ¹H NMR. ¹³C NMR, HR-MS); Diretinoyl AZA tosylate (Compound 4, 78% yield, ¹H NMR. ¹³C NMR, HR-MS).

### Synthesis of Diretinoyl AZA Tosylate (4) as a Representative Following Method C

DCC (0.043 g, 0.21 mmol, 2.1 eq) was dissolved in anhydrous CH₂Cl₂ (1 mL) and then added into a solution of AZA-Tosylate (0.04 g, 0.1 mmol, 1 eq), retinoic acid (0.063 g, 0.21 mmol, 2.1 eq) and DMAP (0.025 g, 0.21 mmol, 2.1 eq) in anhydrous CH₂Cl₂ under Argon with stirring. The reaction was allowed to proceed at room temperature overnight in dark. Crude mixture was filtered, concentrated in vacuo and the residue was passed through a silica column using 9.5:0.5 (v/v) CH₂Cl₂-methanol as eluent. The product was obtained as a yellow solid in 0.0748 g, 0.0776 mmol and 78% yield. ¹H NMR (500 MHz, CDCl₃) δ 7.87 (d, J=8.0 Hz, 2H, phenyl H-2 and H-6), 7.38 (d, J=7.9 Hz, 2H, phenyl H-3 and H-5), 7.35 (s, 1H, imidazole H-5), 7.22 (s, 1H, imidazole H-4), 7.10 (ddd, J=23.8, 15.0, 11.5 Hz, 2H, retinoyl H-5 and H-5′), 6.57 (s, 1H, H-1′), 6.35 (dd, J=15.6, 5.2 Hz, 2H, retinoyl H-4 and H-4′), 6.29-6.13 (m, 6H, retinoyl H-6, H-6′, H-8, H-8′, H-9 and H-9′), 5.79 (s, 1H, retinoyl H-2), 5.54 (s, 1H, retinoyl H-2′), 5.47 (s, 1H, H-2′), 5.09 (s, 1H, H-3′), 4.43-4.32 (m, 2H, retinoyl H-5 and H-5′), 2.47 (s, 3H, phenyl CH3), 2.38 (s, 2×CH₃ ppm; ¹³C NMR (176 MHz, CDCl3) δ 165.29 (retinoyl C-1, C═O), 164.56 (retinoyl C-1, C═O), 156.67 (retinoyl C-3), 156.45 (retinoyl C-3′), 144.11 (imidazole C-2), 140.96 (retinoyl, C-7), 140.80 (retinoyl, C-7′), 137.62 (cyclohexene, C-1), 137.59 (cyclohexene C-1′), 137.10 (retinoyl C-8), 137.04 (retinoyl C-8′), 134.32 (retinoyl C-4), 134.12 (retinoyl C-4′), 132.69 (retinoyl C-5), 132.58 (retinoyl C-5′), 130.33 (cyclohexene C-2), 130.25 (cyclohexene C-2′), 129.46 (retinoyl C-6), 129.32 (retinoyl C-6′), 129.26 (retinoyl C-9), 129.15 (retinoyl C-9′), 128.40 (imidazole C-4), 122.22 (imidazole C-4), 115.55 (retinoyl C-2), 115.45 (retinoyl C-2′), 93.45 (C-1′), 82.46 (C-5′), 81.47 (C-4′), 80.64 (C-2′), 75.61 (C-3′), 39.59 (cyclohexene C-5), 39.57 (cyclohexene C-5′), 34.24 (cyclohexene C-6), 34.23 (cyclohexene C-6′), 33.10 (cyclohexene C-3), 33.02 cyclohexene C-3′), 28.93 (cyclohexene C-6, C-6′), 21.73 (cyclohexene C-2 CH₃), 21.71 cyclohexene C-2′ CH3), 19.18 (cyclohexene C-4), 19.17 (cyclohexene C-4′), 14.19 (retinoyl C-3 CH₃), 14.07 (retinoyl C-3′ CH₃), 12.94 (retinoyl C-6 and C-6′ CH3) ppm; ¹⁹F NMR (376 MHz, CDCl₃) δ −227.57 (td, JF-H-5′=47.1, JF-H-4′=18.7 Hz) ppm; HR-MS (ESI): m/z: 834.4465 [M+Na]+).

Diretinoyl FAZA (Compound 2) and Diretinoyl IAZA (Compound 3) are Other Examples that were Synthesized Using Method C. Characterization Data for Compound 2 are Described Below.

Compound 2:

¹H NMR (700 MHz, CDCl₃) δ 7.38 (d, J=1.3 Hz, 1H imidazole, H-5), 7.20 (d, J=1.2 Hz, 1H imidazole, H-4), 7.05 (ddd, J=18.9, 15.0, 11.4 Hz, 2H, retinoyl H-5 and H-5′), 6.71 (d, J=1.4 Hz, 1H, H-1′), 6.29 (dd, J=15.6, 5.1 Hz, 3H, retinoyl H-4, H-6, H-6′), 6.22 (d, J=14.9 Hz, 1H, retinoyl H-4′)), 6.18-6.10 (m, 4H, retinoyl H-8, H-8′, H-9, H-9′), 5.80 (s, 1H, retinoyl H-2), 5.54 (s, 1H, retinoyl H-2′), 5.49 (s, 1H, H-2′), 5.16 (s, 1H, H-3′), 4.74-4.72 (m, 1H, H-4′), 4.70-4.65 (m, 2H, H-5′), 2.34 (s, 3H, retinoyl C-3 CH₃), 2.29 (s, 3H, retinoyl C-3′ CH₃), 2.02-1.98 (m, 4H, cyclohexene H-3, H-3′), 2.00 (s, 3H, retinoyl C-7 CH3), 1.99 (s, 3H, retinoyl C-7′ CH₃), 1.70 (s, 3H, cyclohexene C-2 CH₃), 1.69 (s, 3H, cyclohexene C-2′ CH₃), 1.64-1.57 (m, 4H, cyclohexene H-4, H-4′), 1.49-1.43 (m, 4H, cyclohexene H-5, H-5′), 1.33-1.22 (m, 3H), 1.01 (s, 12H, cyclohexene C-6 2×CH₃ and C-6′ 2×CH₃ ppm; 13C NMR (176 MHz, CDCl₃) b 165.29 (retinoyl C-1, C═O), 164.56 (retinoyl C-1, C═O), 156.67 (retinoyl C-3), 156.45 (retinoyl C-3′), 144.11 (imidazole C-2), 140.96 (retinoyl, C-7), 140.80 (retinoyl, C-7′), 137.62 (cyclohexene, C-1), 137.59 (cyclohexene C-1′), 137.10 (retinoyl C-8), 137.04 (retinoyl C-8′), 134.32 (retinoyl C-4), 134.12 (retinoyl C-4′), 132.69 (retinoyl C-5), 132.58 (retinoyl C-5′), 130.33 (cyclohexene C-2), 130.25 (cyclohexene C-2′), 129.46 (retinoyl C-6), 129.32 (retinoyl C-6′), 129.26 (retinoyl C-9), 129.15 (retinoyl C-9′), 128.40 (imidazole C-4), 122.22 (imidazole C-4), 115.55 (retinoyl C-2), 115.45 (retinoyl C-2′), 93.45 (C-1′), 82.46 (C-5′), 81.47 (C-4′), 80.64 (C-2′), 75.61 (C-3′), 39.59 (cyclohexene C-5), 39.57 (cyclohexene C-5′), 34.24 (cyclohexene C-6), 34.23 (cyclohexene C-6′), 33.10 (cyclohexene C-3), 33.02 cyclohexene C-3′), 28.93 (cyclohexene C-6, C-6′), 21.73 (cyclohexene C-2 CH3), 21.71 cyclohexene C-2′ CH3), 19.18 (cyclohexene C-4), 19.17 (cyclohexene C-4′), 14.19 (retinoyl C-3 CH3), 14.07 (retinoyl C-3′ CH3), 12.94 (retinoyl C-6 and C-6′ CH3) ppm; 19F NMR (376 MHz, CDCl₃) δ −227.57 (td, JF-H-5′=47.1, JF-H-4′ =18.7 Hz) ppm; HR-MS (ESI): m/z: 834.4465 [M+Na]+).

### Class 2: 2′-O-Substituted Esters of 5′-Halo α/β-AZAs

### Examples of the Compound Synthesized Under this Class Include 5′-Fluoro-2′-O-Retinoylarabinofuranosyl-2-Nitroimidazole (Monoretinoyl FAZA, Compound 5) and 5′-Lodo-2′-O-Retinoylarabinofuranosyl-2-Nitroimidazole (Monoretinoyl IAZA, Compound 6)

Synthesis of monoretinoyl IAZA (6) as a representative of this class is described.

### Step 1: Synthesis of 3′,5′-O,O-tetraisopropyldisilanoxyl-α-AZA (TIPS-α-AZA)

Tetraisopropyl disiloxane dichloride (0.444 g, 450 uL, 1.41 mmol, 1.17 eq) was added into a solution of AZA (0.294 g, 1.2 mmol, 1 eq) in anhydrous. pyridine (4 mL) and the reaction was allowed to proceed overnight at room temperature. The mixture was washed with copper sulfate solution and extracted in ethyl acetate. The combined organic layers were dried over anhydrous, sodium sulfate, evaporated in vacuo and the residue was purified via column chromatography using 9.5:0.5 CH₂Cl₂-methanol as eluent to give 0.4132 g (0.847 mmol and 71% yield) of the TIPS-AZA.

### Step 2: Synthesis of 3′,5′-O,O-tetraisopropyldisilanoxyl-2′-O-retinoyl-α-AZA (Monoretinoyl TIPS-α-AZA)

TIPS-α-AZA (0.4132 g, 0.847 mmol, 1 eq), retinoic acid (0.280 g, 0.9317 mmol, 1.1 eq) and DMAP (0.114 g, 0.9317 mmol, 1.1 eq) were dissolved in 15 mL anhydrous. CH₂Cl₂. DCC (0.192 g, 0.9317 mmol, 1.1 eq) in 5 mL anhydrous CH₂Cl₂ was added into the resulting solution and the reaction proceeded at room temperature overnight in dark under Argon. After completion, the crude mixture was filtered, evaporated to dryness and the residue was purified via column chromatography using 9.5:0.5 CH₂Cl₂— methanol as eluent to give 0.5635 g, (0.73 mmol, 86% yield) of monoretinoyl TIPS-α-AZA; ¹H NMR (400 MHz, CDCl₃) δ 7.51 (d, J=1.3 Hz, 1H, imidazole, H-5), 7.20 (d, J=1.2 Hz, 1H, imidazole, H-4), 7.06 (dd, J=15.0, 11.4 Hz, 1H, retinoyl H-5), 6.69 (d, J=3.6 Hz, 1H, H-1′), 6.29 (dd, J=15.1, 10.1 Hz, 2H, retinoyl H-4, and H₆), 6.23-6.10 (m, 2H, retinoyl H-8 and H-9), 5.79 (s, 1H, retinoyl H-2), 5.53 (dd, J=5.6, 3.6 Hz, 1H, H-2′), 4.61 (dd, J=7.2, 5.5 Hz, 1H, H-3′), 4.22 (td, J=6.4, 3.3 Hz, 1H, H-4′), 4.08 (dd, J=12.4, 3.4 Hz, 1H), 3.97 (dd, J=12.4, 6.0 Hz, 1H), 2.30 (s, 3H), 2.02 (d, J=6.6 Hz, 7H), 1.72 (s, 4H), 1.62 (ddt, J=9.1, 6.4, 4.0 Hz, 4H), 1.52-1.40 (m, 3H), 1.17-0.94 (m, 28H) 1.03 (s, cyclohexene C-6, CH₃) ppm; m/z: 792.4057 [M+Na]+.

### Step 3: Synthesis of 2′-O-retinoyl-α-AZA (Monoretinoyl-α-AZA)

3′,5′-O-TIPS-2′-O-retinoyl AZA (0.5635 g, 0.73 mmol 1 eq) was dissolved in dry THF (2 mL) and then tetrabutylammonium fluoride (2.03 mmol, 2.78 eq) was added. The resulting mixture was stirred overnight at room temperature in dark. After reaction completion, the reaction mixture was concentrated in vacuo, and passed through a silica column using 9.5:0.5 CH₂Cl₂-methanol as eluent to give 0.275 g (0.52 mmol; 71%) of pure 2′-O-retinoyl AZA in 71% yield. ¹H NMR (400 MHz, CDCl₃) δ 7.52 (d, J=1.2 Hz, 1H, imidazole, H-5), 7.19 (d, J=1.2 Hz, 1H, imidazole, H-4), 7.09 (dd, J=15.0, 11.4 Hz, 1H, retinoyl H-5), 6.74 (d, J=2.2 Hz, 1H, H-1), 6.31 (dd, J=15.6, 10.1 Hz, 2H′, retinoyl H-4, and H₆), 6.20-6.11 (m, 2H, retinoyl H-8 and H-9), 5.82 (s, 1H, retinoyl H-2), 5.22 (t, J=2.5 Hz, 1H, H-2′), 4.48 (td, J=5.1, 3.8 Hz, 1H, H-3′), 4.38 (dd, J=5.0, 2.8 Hz, 1H, H-4′), 3.94-3.78 (m, 2H, H-5′), 2.35 (s, 3H, retinoyl C-3, CH₃), 2.07-1.96 (m, 5H, cyclohexene H-3, retinoyl C-7 CH₃), 1.72 (s, 3H, C-2 CH₃), 1.68-1.57 (m, 2H, cyclohexene H-4), 1.51-1.43 (m, 2H, cyclohexene H-5), 1.04 (s, cyclohexene C-6, CH₃) ppm; ¹³C NMR (126 MHz, CDCl₃) δ 166.69 (retinoyl C-1, C═O), 156.89 (retinoyl C-3), 144.42 (imidazole C-2), 141.21 (retinoyl, C-7), 137.82 (cyclohexene C-1), 137.26 (retinoyl C-8), 134.42 (retinoyl C-4), 132.97 (retinoyl C-5), 130.55 (cyclohexene C-2), 129.69 (retinoyl C-6), 129.41 (retinoyl C-9), 128.72 (nitroimidazole C-4), 122.78 (nitroimidazole C-5), 115.81 (retinoyl C-2), 92.12 (C-1′), 87.94 (C-2′), 85.57 (C-3′), 77.16 (C-4′), 62.24 (C-5′), 39.80 (cyclohexene C-5), 34.46 (cyclohexene C-6), 33.32 (cyclohexene C-3), 29.15 (cyclohexene C-6 CH₃), 21.94 (cyclohexene C-2 CH₃), 19.39 (cyclohexene C-4), 14.37 (retinoyl C-3 CH₃), 13.17 (retinoyl C-7 CH₃) ppm; m/z: 550.2533 [M+Na]+.

### Step 4: Synthesis of 5′-Iodo-2′-O-retinoylarabinofuranosyl-2-nitroimidazole (Compound 6)

2′-O-Retinoyl AZA (0.0528 g, 0.1 mmol, 1 eq) and triphenylphosphine (0.0532 g, 0.203 mmol, 2.03 eq) were dissolved in anhydrous pyridine (5 mL). After stirring for 5 mins, iodine (0.0512 g, 0.203 mmol, 2.03 eq) was added and the resulting mixture was stirred at RT and monitored after 6 h. Methanol was then added to quench the reaction, washed with water and extracted with ethyl acetate. The combined organic extracts were dried over anhydrous sodium sulfate, filtered and evaporated. Crude residue was passed through a silica gel column using 9.5:0.5 CH₂Cl₂-methanol as eluent to give 6 in 0.0265 g, 0.42 mmol and 42% yield. ¹H NMR (400 MHz, CDCl3) δ 7.55 (d, 1H, imidazole H-5), 7.14 (d, J=1.2 Hz, 1H, imidazole H-4), 7.08 (dd, J=15.0, 11.4 Hz, 1H, retinoyl H-5), 6.71 (d, J=1.8 Hz, 1H, H-1′), 6.31 (dd, J=15.6, 6.4 Hz, 2H, retinoyl H-4 and H-6), 6.20-6.12 (m, 2H, retinoyl H-8 and H-9), 5.81 (s, 1H, retinoyl H-2), 5.30 (dd, J=4.3, 2.3 Hz, 1H, H-2′), 4.59 (td, J=6.7, 3.2 Hz, 1H, H-3′), 4.34 (dd, J=3.5, 2.2 Hz, 1H, H-4′), 3.39 (dq, J=7.3, 3.8 Hz, 2H, H-5′), 2.34 (s, 3H, retinoyl C-3, CH₃), 2.06-1.95 (m, 5H, cyclohexene H-3, retinoyl C-7 CH₃), 1.72 (s, 3H, C-2 CH₃), 1.65-1.53 (m, 2H, cyclohexene H-4), 1.53-1.42 (m, 2H, cyclohexene H-5), 1.03 (s, cyclohexene C-6, CH₃) ppm; ¹³C NMR (101 MHz, CDCl₃) δ 165.81 (retinoyl C-1, C═O), 156.67 (retinoyl C-3), 144.00 (imidazole C-2), 140.98 (retinoyl, C-7), 137.62 (cyclohexene C-1), 137.07 (retinoyl C-8), 134.25 (retinoyl C-4), 132.75 (retinoyl C-5), 130.34 (cyclohexene C-2), 129.47 (retinoyl C-6), 129.23 (retinoyl C-9), 128.26 (nitroimidazole C-4), 123.02 (nitroimidazole C-5), 115.62 (retinoyl C-2), 92.65 (C-1′), 87.91 (C-2′), 84.25 (C-3′), 78.58 (C-4′), 39.60 (cyclohexene C-5), 34.26 (cyclohexene C-6), 33.13 (cyclohexene C-3), 28.96 (cyclohexene C-6 CH₃), 21.75 (cyclohexene C-2 CH₃), 19.19 (cyclohexene C-4), 14.22 (retinoyl C-3 CH₃), 12.99 (retinoyl C-7 CH₃) ppm; HR-MS (ESI): m/z: 638.1742 [M+H]+, 660.1541 [M+Na]+.

### Class II: 5′-O-Sulfonate Esters of 2′-O-Substituted α/β-AZAs

### Example of this Class of Compounds Includes the Synthesis of 2′-O-Retinoyl-5′-O-toluenesulfonyl α-AZA (2′-O-Retinoyl α-AZA Tosylate, Compound 7)

Characterization data for this molecule are described below.

Data for 2′-O-Retinoyl α-AZA Tosylate (7).

Yield 143 mg (56.5%); ¹H NMR (400 MHz, CDCl₃) δ 7.80 (d, J=8.1 Hz, 2H), 7.44 (s, 1H), 7.35 (d, J=8.0 Hz, 2H), 7.18-7.10 (m, 1H), 7.08 (d, J=11.4 Hz, 1H), 6.56 (d, J=2.1 Hz, 1H), 6.32 (dd, J=15.6, 11.5 Hz, 2H), 6.17 (d, J=16.4 Hz, 2H), 5.76 (s, 1H), 5.20 (t, J=2.3 Hz, 1H), 4.59 (q, J=4.9 Hz, 1H), 4.31 (s, 1H), 4.22 (d, J=5.2 Hz, 2H), 3.50 (s, 1H), 2.45 (s, 3H), 2.34 (s, 3H), 2.06-1.98 (m, 5H), 1.73 (s, 3H), 1.68-1.56 (m, 2H), 1.53-1.38 (m, 2H), 1.04 (s, 6H) ppm; ¹³C NMR (101 MHz, CDCl₃) δ 166.13, 156.94, 145.53, 141.24, 137.79, 137.23, 134.37, 133.00, 132.58, 130.54, 130.14, 129.70, 129.37, 128.54, 128.17, 122.87, 115.65, 114.80, 92.40, 85.18, 84.30, 77.16, 68.05, 39.76, 34.43, 33.29, 29.12, 21.91, 21.85, 19.35, 14.37, 13.15; m/z: 704.2613 [M+Na]⁺.

### Class IV: Substituted Benzo-1,2,4-Triazene-1,4-Dioxides

Examples of the compounds synthesized under this class include 2-(2-haloethoxyethyl)amino-1,2,4-benzotrizene-1,4-dioxide and 2-aminopropanoxy-3-(2-glucosyl-1,3,4,6-tetra-O-acetyl)-1,2,4-benzotriazene-1,4-dioxide and the related derivatives, where X=−OTs, OTf, ONs, OMs, a (radio)halogen, H. Syntheses (Scheme 4 and characterization data for three novel compounds are provided below.

### 3-(2-(2-(Tosyloxy)ethoxy)ethyl)amino-1,2,4-benzotriazene 1,4-dioxide (8)

To a solution of 3-(2-(2-(tosyloxy)ethoxy)ethyl)amino-1,2,4-benzotriazene 1-oxide (2 g, 4.94 mmol) in CH₂Cl₂ (70 mL) was added NaHCO₃ (0.83 g, 9.89 mmol) and m-chloroperbenzoic acid (1.3 g, 7.41 mmol) and the reaction mixture was stirred for 6 h at room temperature. The solvent was evaporated and the residue was partitioned between dilute aqueous NH3 (20 mL) and CH₂Cl₂ (3×70 mL). The organic fraction was dried and the solvent was evaporated. The residue was purified by chromatography (10:1 EtOAc—CH₃OH) to give 8 (730 mg, 35%) as a red solid: Rf 0.38 (10:1 EtOAc—CH₃OH); mp (EtOAc/CH₃OH) 47±2° C.; IR cm−1 3250, 3087, 2985, 2954, 2920, 2874, 1618, 1598, 1495, 1446, 1415, 1357, 1341, 1320, 1246, 1180, 1111, 1091, 1043, 1004; ¹H NMR (400 MHz, CDCl₃, δH) 8.29-8.19 (m, 2H, Ar), 7.82 (ddd, J=8.5, 7.0, 1.2 Hz, 1H, Ar), 7.79-7.71 (m, 2H, Ar), 7.51-7.43 (m, 1H, Ar), 7.34-7.27 (m, 2H, Ar), 4.16-4.09 (m, 2H, CH₂), 3.75-3.54 (m, 6H, CH₂×3), 2.39 (s, 3H, Ar—CH₃); ¹³C NMR (101 MHz, CDCl₃, δC) 149.77, 144.87, 138.27, 135.75, 132.84, 130.49, 129.84, 127.93, 127.27, 121.57, 117.35, 69.23, 69.00, 68.54, 41.05, 21.61. HRMS (ESI) Calcd. for (M+Na)+C₁₈H₂₀N₄O₆SNa: 443.1001. Found: 443.1001.

### 3-(2-(2-Iodoethoxy)ethyl)amino-1,2,4-benzotriazene 1,4-dioxide (I-TPZ) (9)

A solution of 3-(2-(2-(tosyloxy)ethoxy)ethyl)amino-1,2,4-benzotriazene 1-oxide (100 mg, 0.24 mmol) and NaI (106.42 mg, 0.71 mmol) in DMF (2 mL) was heated at 100° C. for 1 h. The solution was quenched with cold H₂O (20 mL) and extracted with CH₂Cl₂ (2×20 mL). The organic layer was concentrated under reduced pressure and the crude residue was purified with chromatography (10:1 EtOAc-CH₃OH) yielding 9 (80.91 mg, 87%) as a red solid: Rf 0.39 (10:1 EtOAc—CH₃OH); IR cm−1 3244, 3109, 2948, 2892, 2851, 1620, 1600, 1493, 1439, 1413, 1386, 1356, 1341, 1255, 1202, 1177, 1133, 1106, 1089, 1032; 1H NMR (400 MHz, CDCl₃, δH) 8.32-8.25 (m, 2H, Ar), 7.84 (ddd, J=8.6, 7.0, 1.2 Hz, 1H, Ar), 7.49 (ddd, J=8.6, 7.0, 1.1 Hz, 1H, Ar), 7.41 (br s, 1H, NH), 3.79 (q, J=5.9, 5.5 Hz, 2H, CH₂), 3.76-3.70 (m, 4H, CH₂×2), 3.24 (t, J=6.6 Hz, 2H, CH₂); ¹³C NMR (101 MHz, CDCl3, δC) 149.77, 138.30, 135.76, 130.51, 127.28, 121.62, 117.43, 71.51, 68.65, 41.19, 2.66. HRMS (ESI) Calcd. for (M+Na)+C₁₁H₁₃ IN₄O₃Na: 398.9930. Found: 398.9928.

### 3-(2-(2-hydroxyethoxy)ethyl)amino-1,2,4-benzotriazene 1,4-dioxide (10)

To a solution of 3-(2-(2-hydroxyethoxy)ethyl)amino-1,2,4-benzotriazene 1-oxide (300 mg, 1.19 mmol) in CH₂Cl₂ (40 mL) was added NaHCO₃ (0.21 g, 2.4 mmol) and m-chloroperbenzoic acid (0.31 g, 1.78 mmol) and the reaction mixture was stirred for 6 h at room temperature. The solvent was evaporated and the residue was partitioned between dilute aqueous NH₃ (20 mL) and CH₂Cl₂ (3×70 mL). The organic fraction was dried and the solvent was evaporated. The residue was purified by chromatography (10:1 EtOAc—CH₃OH) to give 10 (130 mg, 35%) as a red solid: Rf 0.28 (10:1 EtOAc—CH₃OH); mp (EtOAc-CH₃OH) 271±2° C.; ¹H NMR (400 MHz, CDCl₃, δH) 8.21 (ddd, J=8.7, 1.4, 0.5 Hz, 1H, Ar), 7.66 (ddd, J=8.4, 6.9, 1.5 Hz, 1H, Ar), 7.55 (d, J=7.9 Hz, 1H, Ar), 7.31-7.22 (m, 1H, Ar), 6.07 (br s, 1H, NH), 3.84-3.70 (m, 6H, CH₂×3), 3.67-3.58 (m, 2H, CH₂), 2.79 (br s, 1H, OH); ¹³C NMR (125 MHz, CDCl₃, δC); ¹³C NMR (101 MHz, CDCl₃, δC) 158.93, 148.69, 135.56, 130.86, 126.38, 124.90, 120.40, 72.35, 69.63, 61.74, 41.25. Similarly, 3-(2-(2-retinoylethoxy)ethyl)amino-1,2,4-benzotriazene 1,4-dioxide (10a, Retinoyl-TPZ) and 3-(2-(2-azidoethoxy)ethyl)amino-1,2,4-benzotriazene 1,4-dioxide (10b, A-TPZ) were also synthesized and fully characterized. Data for 10a. HRMS (ESI) Calcd. for (M+Na)⁺ C₃₁ H₄₀ N₄ Na O₅: 571.2891. Found: 571.2883. HRMS (ESI) Calcd. for (M+H)⁺ C₃₁ H₄₁ N₄ O₅: 549.3071. Found: 549.3076. Data for 10b. HRMS (ESI) Calcd. for (M+Na)⁺ C₁₁ H₁₃ N₇ Na O₃: 314.0972. Found: 314.0974. HRMS (ESI) Calcd. for (M+H)⁺ C₁₁ H₁₄ N₇ O₃: 292.1153. Found: 292.1175.

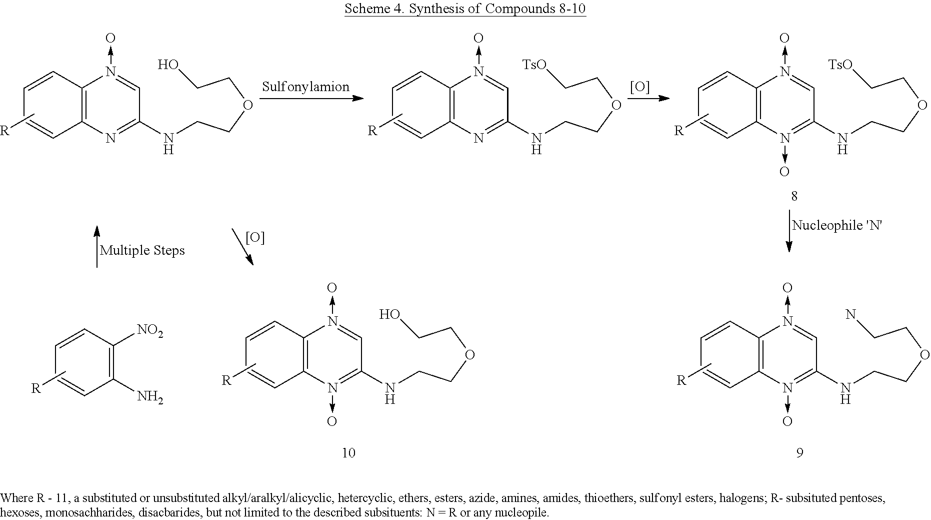

General Formula for the Radiopharmaceuticals synthesized under this class is provided in Scheme 5, below.

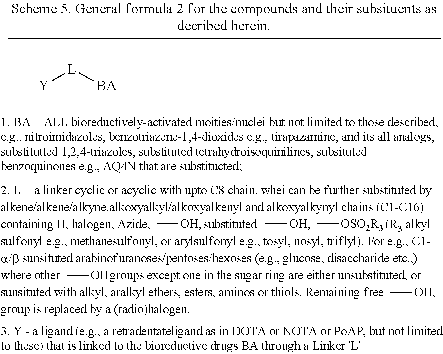

### Class V: Sugar-Conjugated Benzotriazene-1,4-Dioxides

### Subclass V.1. Glucose 6-Conjugated benzotriazene-1,4-dioxides

Various categories of glucose 6-conjugated benzotriazene-1,4-dioxide molecules are provided below.

### Category V.1.1. This Class of Compounds Contain the Molecules where Benzotriazene-1,4-Dioxide Moiety is Conjugated to Various Sugar Moieties Through a Linker Having a (Radio)Theranostic Moiety as Shown in Scheme 6

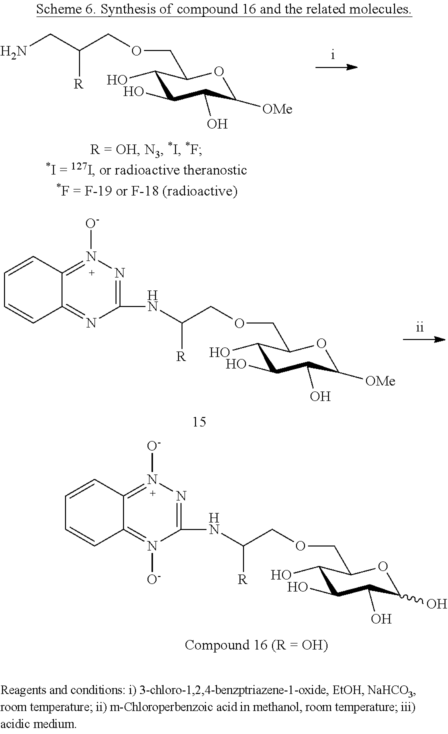

### Example of the Representative Molecules 15 and 16 Synthesized Under this Category is Described

### 1-α-D-O-Methyl 6-O-(9-[2-amino-1,2,4-benzotriazene-1-oxide]-8S-O-acetyl-propyl)-glucopyranose (15)

1-α-D-O-Methyl-6-O-(3[2-hydroxy]aminopropyl)-glucose.hydrochloride (0.255 g) and 3-chloro-1,2,4-benzotriazene-1-oxide (1.5 equivalent) were dissolved in ethanol and reacted in presence of sodium bicarbonate (3 equivalent) for 9 days at room temperature. Tirapazamine-glucose conjugated monoxide product 15 was obtained as a bright yellow solid in 36% yield (0.207 g) after column purification, and subjected to oxidation as described below.

### 1-α-D-6-O-(9-[2-amino-1,2,4-benzotriazene-1,4-dioxide]-8R/8S-hydroxypropyl)-glucopyranose (16)

Monoxide product obtained above was treated with 1.3 equivalent of m-chloroperbenzoic acid in methanol for 17 h at room temperature to afford the corresponding 1,4-dioxide product, which was demethylated in acidic medium to afford final product 16in ˜40% overall yield (96.41% pure by HPLC). ¹H-NMR (CD₃OD)−δ 3.1-3.46 (mixed m, 5H, H-2, H3, H-4 and H7 and H-7′ of propyl chain), 3.6-3.93 (multiple m, 5H, 2×H-6, 2×H9′, 1H-8′), 4-3-4.6 (mergeed m, 1H-H-1), 7.59 and 8.0 (two m, each for 1H, H6 and H-7 of phenyl), 8.18 and 8.31 (two d, H5 and H-8 of phenyl); Elemental analysis for C₁₆H₂₂N₄O₉.7/5H₂O, Calcd C, 43.72%, H5.69%, N, 12.75%; found C, 43.98%, H, 5.63%, N, 12.05% . . . MS (ES+)−M+1 (415.12)−abundance (100%).

### Category V.1.2. Molecules Synthesized Under this Class Include Various Sugars that are Conjugated to Benzotriazene-1,4-Dioxides Through a Linker, and a (Radio)Theranostic Arm is Further Substituted to this Linker

Examples of two representative molecules synthesized under this class are provided in Scheme 7 and Scheme 8, below, and the synthesis conditions are specified.

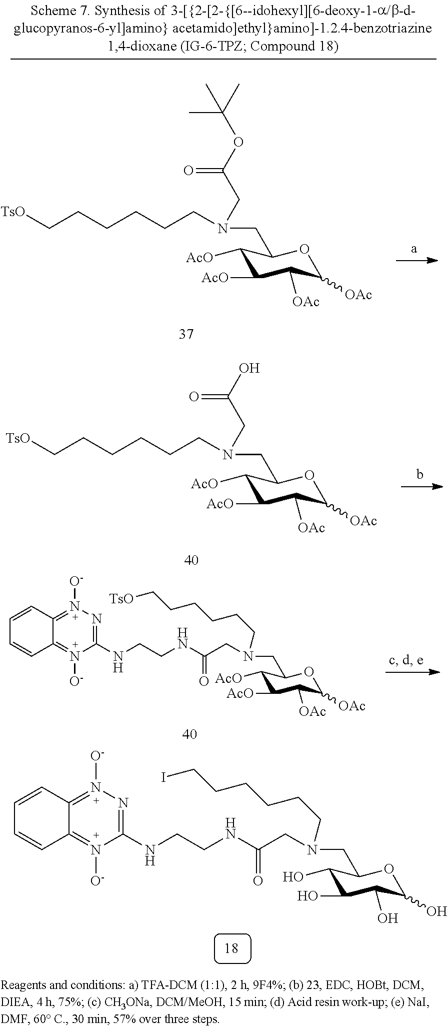

‘IG-6-TPZ’ theranostic (compound 18) is synthesized following the reaction method described in Scheme 3. HRMS (ESI) Calcd. for (M+Na)⁺ C₂₃H₃₅IN₆Na O₈: 673.1453. Found: 673.1459. HRMS (ESI) Calcd. for (M+H)⁺ C₂₃H₃₆IN₆O₈: 651.1634. Found: 651.1644.

### Subclass V.2. Glucose 2-Substituted benzotiazene-1,4-dioxides

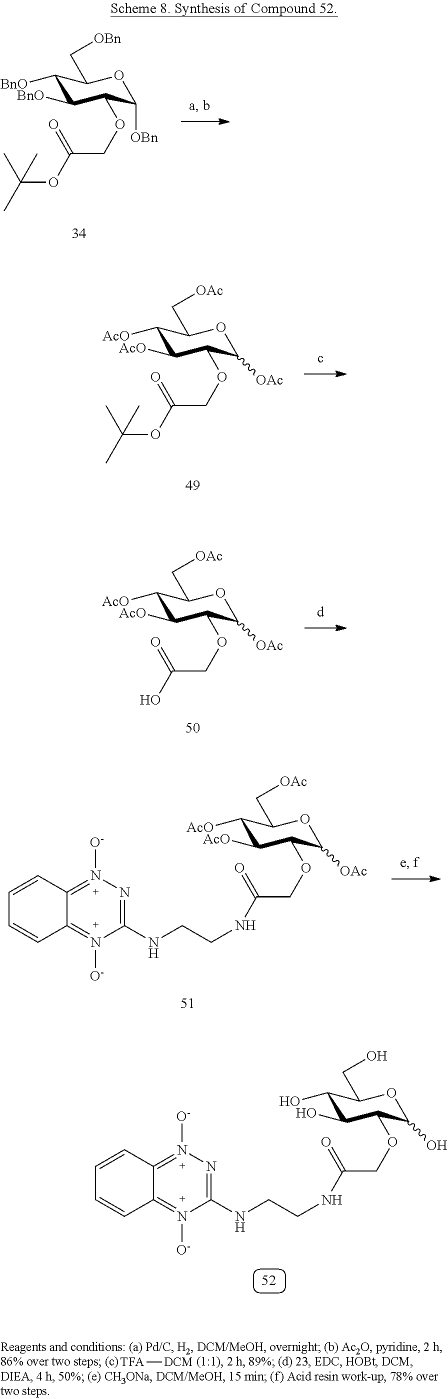

As a representative molecule, 3-[{2-[2-{[6-iodohexyl][2-(1-α/β-d-glucopyranos-3-O-yl)ethyl]amino} acetamido]ethyl}amino]-1,2,4-benzotriazine 1,4-dioxide (IG-2-TPZ; compound 52) is synthesized following the reaction method described in Scheme 5; HRMS (ESI) Calcd. for (M+Na)⁺ C₁₇ H₂₃ N₅ Na O₉: 464.1388. Found: 464.1384.

### Subclass V.3. Synthesis of Gluc-2 Conjugated TPZ with a (Radio)Theranostic Arm′

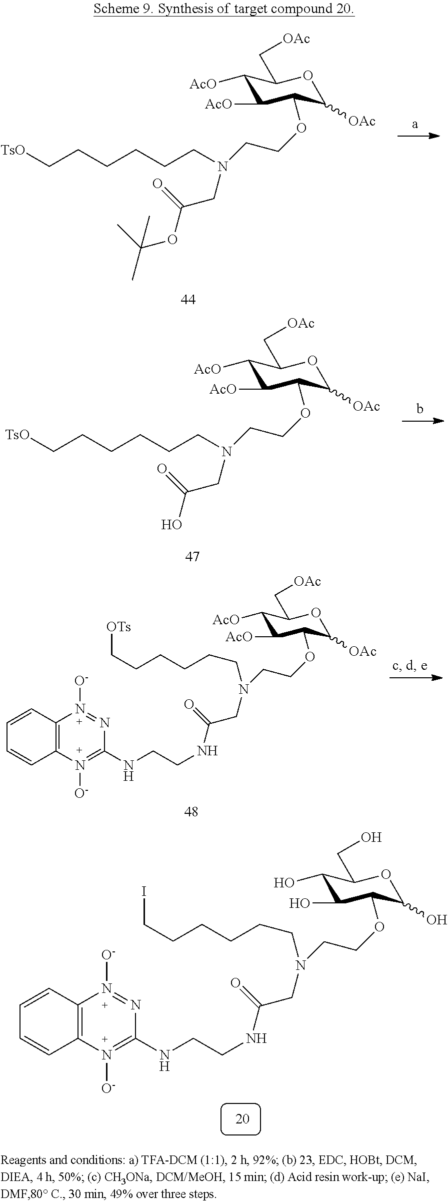

Characterization data for ‘IG-2-TPZ’ theranostic (compound 20) is synthesized following the reaction method described in Scheme 7; HRMS (ESI) Calcd. for (M+Na)⁺ C₂₅ H₃₉ I N₆ Na O₉: 717.1715. Found: 717.1715. HRMS (ESI) Calcd. for (M+H)⁺ C₂₅ H₄₀ I N₆ O₉: 695.1896. Found: 695.1889.

### Class VI: DOTA-AZA Class of Drugs

DOTA-AZA pivaloylate was synthesized under this class of drugs and fully characterized. Characterization data are described below and the stepwise synthesis process is shown in Schemes 10 and Scheme 11.

Dipivaloyl-AminoAZA (DPAZANH2; Compound 11):

¹H NMR (400 MHz, CDCl₃) δ 7.38 (d, J=1.0 Hz, 1H, imidazole, H-5), 7.22 (d, J=1.0 Hz, 1H, imidazole, H-4), 6.62 (d, J=1.5 Hz, 1H, H-1′), 5.35 (dd, J=1.5, 1.0 Hz, 1H, H-2′), 4.98 (dd, J=2.0, 1.0 Hz, 1H, H-3′), 4.41 (td, J=2.5, 6.1 Hz, 1H, H-4′), 3.10-3.01 (m, 2H, H-5′), 1.28 (s, 9H, 3×CH₃), 1.09 (s, 9H, 3×CH₃) ppm; ¹³C NMR (125 MHz, CDCl₃) δ 177.16 (C═O), 176.25 (C═O), 128.48 (imidazole, C-4), 122.09 (imidazole, C-5), 93.28 (C-1′), 90.58 (C-5′), 81.71 (C-4′), 44.05 (CH₂), 38.79 (pivaloyl, C), 38.61 (pivaloyl C), 26.94 (CH₃), 26.86 (CH₃) ppm; HR-MS (ESI): m/z: 413.2036 [M+H]+DOTA-AZA Conjugate (DOTA-DPAZA; Compound 12): ¹H NMR (400 MHz, CDCl₃) δ 7.55 (s 1H, imidazole, H-5), 7.20 (s, 1H, imidazole, H-4), 6.59 (s, 1H, H-1′), 5.36 (s, 1H, H-2′), 4.96 (s, 1H, H-3′), 4.68 (td, J=8.0, 4.0 Hz, 1H, H-4′), 3.7-1.8 (m, 26H, 13×CH₂), 1.48 (s, 9H, 3×CH₃), 1.47 (s, 18H 6×CH₃) 1.28 (s, 9H, 3×CH₃), 1.08 (s, 9H, 3×CH₃) ppm; ¹³C NMR (100 MHz, CDCl₃) δ 176.82 (C═O), 176.18 (C═O), 172.52 (C═O), 172.25 (C═O), 143.72 (imidazole, C-5), 128.48 (imidazole, C-4), 122.09 (imidazole, C-5), 93.23 (C-1′), 90.58 (C-5′), 81.88 (CH₂), 81.81 (CH₂), 81.78 (CH₂), 81.45 (CH₂), 55.94 (C), 55.84 (C), 55.69 (C), 41.55 (CH₂), 38.70 (pivaloyl, C), 38.52 (pivaloyl C), 28.01 (CH₃), 28.79 3 (CH₃), 26.88 (CH₃) 28.80 (CH₃) ppm; HR-MS (ESI): m/z: 967.5728 [M+H]+, 989.5528 [M+Na]+.

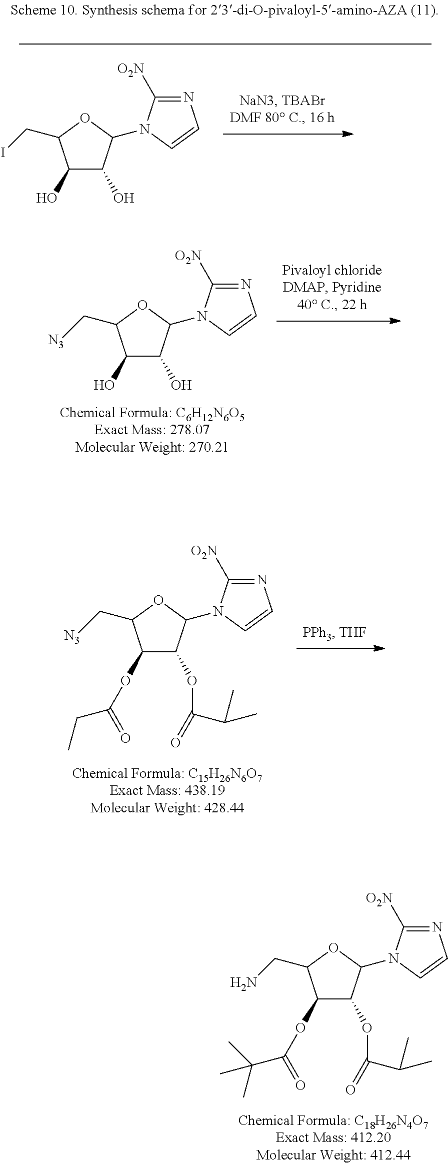 [SMILES: O=[N+]([O-])c1nccn1C1OC(CI)[C@H](O)[C@H]1O ; [N-]=[N+]=NCC1OC(n2ccnc2[N+](=O)[O-])[C@H](O)[C@H]1O ; CCC(=O)O[C@H]1C(CN=[N+]=[N-])OC(n2ccnc2[N+](=O)[O-])[C@@H]1OC(=O)C(C)C ; CC(C)C(=O)O[C@H]1C(n2ccnc2[N+](=O)[O-])OC(CN)[C@@H]1OC(=O)C(C)(C)C]

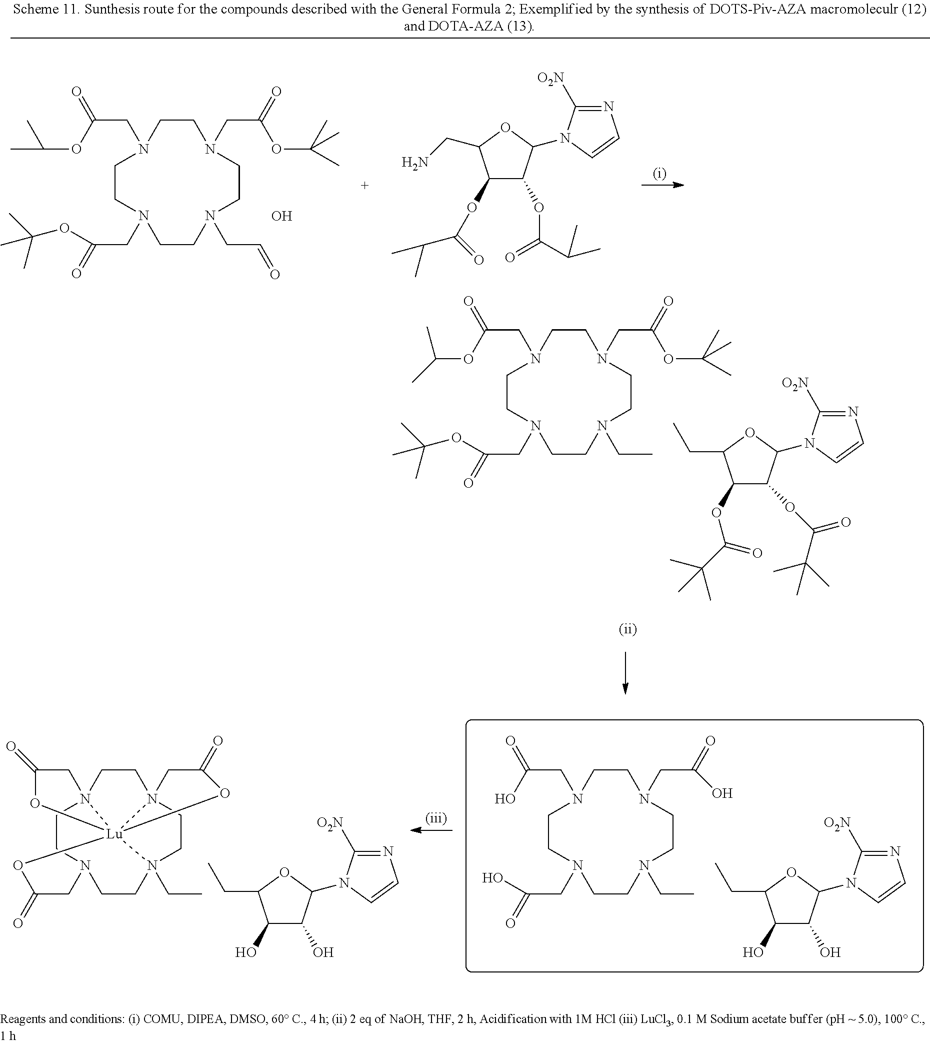

### Class VII: DOTA-TPZ-Based Drugs

This class of compounds relate to the TPZ and other bioreductively activated molecules to a chelating macrocyclic ligand e.g., DOTA, NOTA, but the claim is not limited to these ligands. As an example, the synthesis process and characterization data for DOTA-TPZ molecule Compound 14 are provided below (Scheme 12).

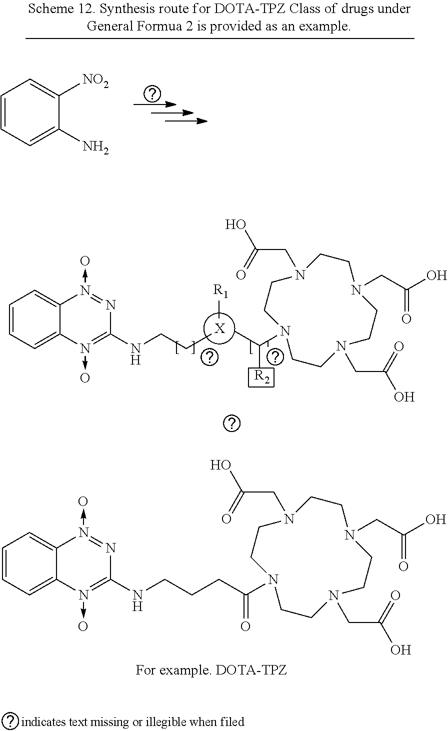

Brief methodology and the characterization data for DOTA-TPZ: Tirapazamine carboxylate advanced intermediate was coupled with DOTA using EDC¬-HOBt as a coupling agent and diisopropylethyl amine as a base. Then, the carboxylate groups of DOTA were hydrolysed using TFA, trifluoroacetic acid to yield TPZ-DOTA, 14, as a red powder. ¹H NMR (500 MHz, CD₃OD) δ 8.30 (dt, J=8.9, 1.6 Hz, 1H), 8.16 (ddd, J=8.7, 4.3, 1.1 Hz, 1H), 7.98 (ddt, J=8.2, 7.0, 1.2 Hz, 1H), 7.58 (dddd, J=8.4, 7.0, 2.7, 1.2 Hz, 1H), 4.86 (s, 4H), 3.91-2.83 (m, 20H), 2.58 (t, J=7.1 Hz, 1H), 2.43 (t, J=7.3 Hz, 1H), 2.01 (tt, J=9.3, 6.4 Hz, 2H); ¹³C NMR (125 MHz, CD₃OD) δ 161.71, 161.44, 150.29, 138.34, 136.52, 136.33, 130.67, 126.83, 121.11, 116.18, 54.53, 46.45, 40.37, 40.31, 30.64, 29.75, 24.22, 24.12.). HRMS (ESI) Calcd. for (M−H)−C₂5H₃5N₈O₉: 591.2538. Found 591.2538.

### Class VII—Radiopharmaceuticals and their Compositions

Examples of this class of radiopharmaceuticals include the molecules described in General formula 1 (Scheme 13) where a radiohalogen or any other radioisotope is also present in the molecule, as in F-18 labelled dipivaloyl 5′-¹⁸FAZA and I-123/124/125/131-labelled diretinoyl-^{123/124/125/131}IAZA, radiolabeled ret-IAZA and retinoyl FAZA; for both α- and β-conformers.

Radiolabeling method, IAZA, Ret-IAZA, But-IAZA, Ret-FAZA, But-FAZA radiopharmaceuticals and their compositions will be described in full patent application. Example schematic of the radiosynthesis methodology for this class of compounds is described below.

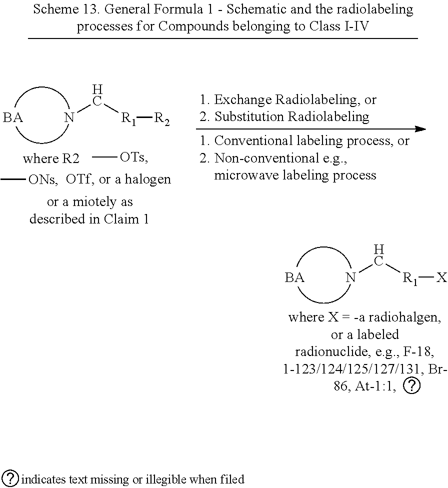

### Example 1: 2′-O-Retinoyl [¹³¹I]IAZA (Compound [¹³¹I]I-6)

Radiolabeling:

2′-O-Retinoyl [¹³¹I]IAZA (100 μg), pre-dissolved in anhydrous ethanol (100 μL), is added to the vial containing preweighed amount of pivalic acid (3.5 mg±5%). The contents are gently swirled until the solution becomes clear and transferred to the reaction vial containing radioiodide. The reaction vial is then placed on a pre-heated block (50±5° C.), and the solvent is slowly evaporated by a gentle stream of nitrogen through the solution until dryness (melt is formed). Radiolabeling is performed for min at this temperature and then vial is cooled down to room temperature prior to purification.

Cartridge Purification:

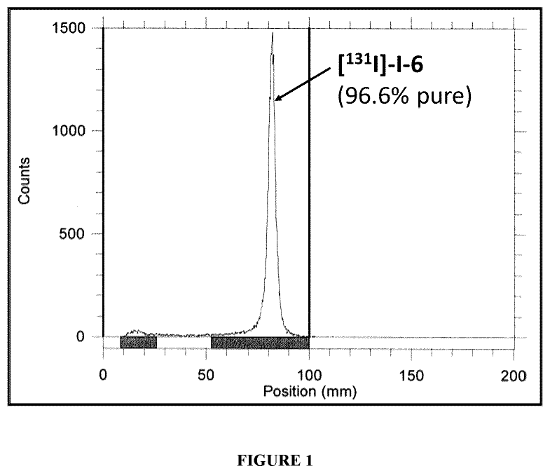

Labelled melt is dissolved in 100 μL of solvent (70% EtOH in sterile water), the vial is gently swirled, and then the contents are withdrawn in a 1 mL syringe. The contents are loaded on preconditioned assembly of two Sep-Pak cartridges, followed by a slow wash with sterile water (30 mL) to remove unreacted iodine from the reaction mixture. Lastly, the cartridge is eluted with USP ethanol (2 mL) and the product is collected in a sterile ‘Product vial’. This process afforded >95% pure 2′-O-Retinoyl [¹³¹I]IAZA in 40-50% radiochemical yield as shown in the radiochromatogram provided below (FIG. 1). The eluted material can be further recomposed with sterile water or saline that is suitable for animal and human subjects, and acceptable by the regulatory authorities.

FIG. 1 Depicts TLC of Purified 2′-O-Retinoyl [¹³¹I]IAZA.

### Example 2: Acetylated [131I]IGAZ

Radiolabeling:

Acetyl-IGAZ (100 μg), pre-dissolved in anhydrous acetonitrile (100 μL), is added to the vial containing pre-weighed amount of pivalic acid (3.5 mg±5%). The contents are gently swirled until the solution becomes clear, and transferred to the reaction vial containing radioiodide. The reaction vial is then placed on a pre-heated block (40±5° C.), and the solvent is slowly evaporated by a gentle stream of nitrogen through the solution until dryness (melt is formed). Once the solvent is removed, and dry ‘melt’ is formed, the reaction vial is removed from the heater. Temperature of the heater is raised (80±5° C.). Once the temperature is stabilized, the reaction vial is replaced on the heater, radiolabeling is performed for 80 min at this temperature, and then vial is cooled down to room temperature prior to purification.

Cartridge Purification:

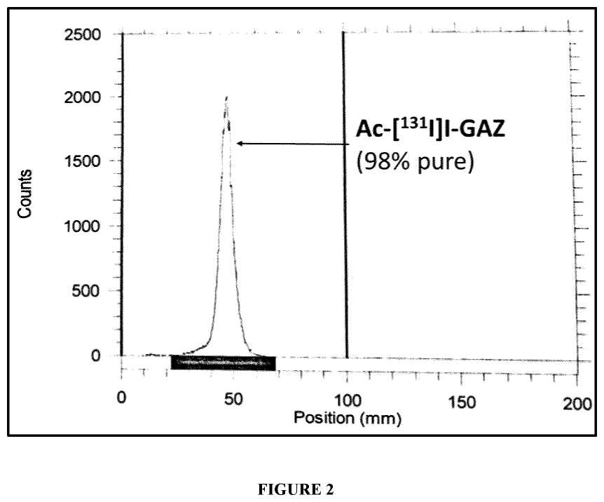

Labelled melt is dissolved in 100 μL of solvent (70% EtOH in sterile water), the vial is gently swirled, and then the contents are withdrawn in a 1 mL syringe. The contents are loaded on preconditioned assembly of two Sep-Pak cartridges, followed by a slow wash with sterile water (10 mL) to remove unreacted iodine from the reaction mixture. Lastly, the cartridge is eluted with USP ethanol (2 mL) and the product is collected in a sterile ‘Product vial’. This process afforded >95% pure acetylated [¹³¹I]IGAZ in 40-50% radiochemical yield as shown in the radiochromatogram provided below (FIG. 2). The eluted material can be further recomposed with sterile water or saline that is suitable for animal and human subjects, and acceptable by the regulatory authorities.

FIG. 2 Depicts Radiochromatogram of Pure Acetyl [¹³¹I]I-GAZ.

### Example 3: [¹³¹I]I-TPZ Radiopharmaceutical (Compound [¹³¹I]-9)

Radiolabeling:

HE-B-23 (100 μg), pre-dissolved in acetonitrile (100 μL), is added to the vial containing radioiodide (V-vial) and then placed on a pre-heated block (80±5° C.). Radiolabeling is performed for 30 min at this temperature and then vial is cooled down to room temperature prior to purification.

Cartridge Purification:

Labelled mixture was taken in 10 μL of acetonitrile, the vial was gently swirled to dissolve the contents, and then the contents were withdrawn in a 1 mL syringe. The contents were loaded on a Waters alumina cartridge that had been preconditioned with USP-grade ethanol (10 mL), followed by sterile water (10 mL). An additional 1 mL sterile water or sterile saline was added to the reaction vial, the whole solution was withdrawn into a sterile syringe, the syringe was attached to the alumina cartridge (preloaded with the labelled product), the contents were slowly pushed through the cartridge and the eluted volume was collected in a sterile ‘Product vial’. This process afforded >95% pure ¹³¹I-B-23 in 40-50% radiochemical yield as shown in the radiochromatogram provided below (FIG. 8).

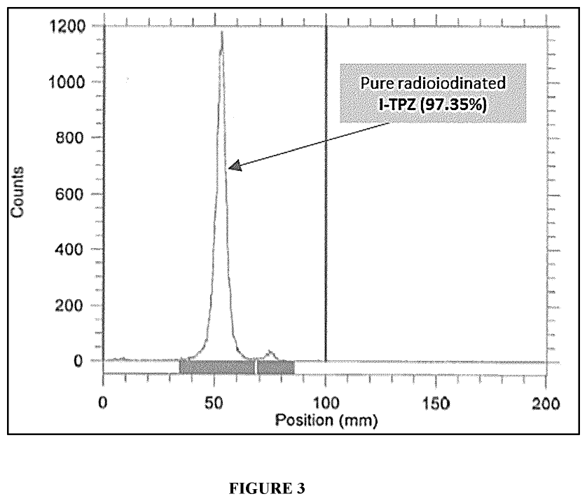

FIG. 3 depicts Radio-TLC of purified [¹³¹I]I-TPZ after passing through alumina cartridge.

### Example 4 Depicts Glucose-6-Substituted [¹³¹I]IG-6-TPZ Radiopharmaceutical

Radiolabeling:

HE-B-129 (100 μg), pre-dissolved in acetonitrile (100 μL), was added to the reacti-vial containing radioiodide (V-vial) and then the vial is placed on the pre-heated block (60±5° C.). Radiolabeling is performed for 30 min at this temperature. The vial is removed, cooled down to room temperature, and then the reaction mixture is purified by solid phase technique as below.

Sep-Pak Purification:

Labelled mixture was taken in 10 μL of acetonitrile, the vial was gently swirled to dissolve the contents, and then the contents were withdrawn in a 1 mL syringe. The contents were loaded on a Waters alumina cartridge that had been preconditioned with USP-grade ethanol (10 mL), followed by sterile water (10 mL). An additional 1 mL sterile water or sterile saline was added to the reaction vial, the whole solution was withdrawn into a sterile syringe, the syringe was attached to the alumina cartridge (preloaded with the labelled product) and the contents were slowly pushed to elute pure labelled product, which was collected in a sterile ‘Product vial’. This process afforded >97% pure ¹³¹I-B-129.

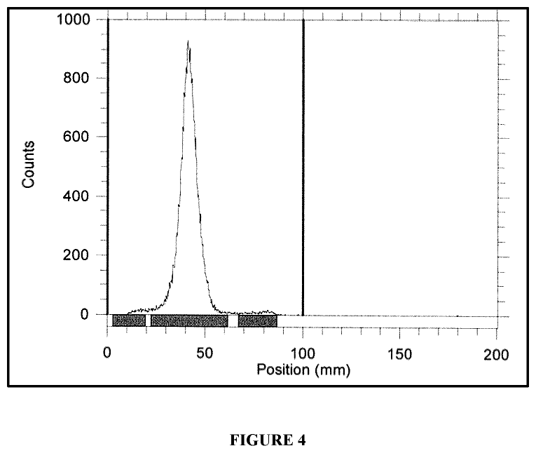

FIG. 4 Depicts a Radiochromatogram of [¹³¹I]IG-6-TPZ after alumina cartridge facilitated solid phase purification.

### Class VIII

General formula 4 for radioligand-based radiopharmaceuticals. Examples of this class of radiopharmaceuticals include where the molecules are chelated with an imaging or radiotherapeutic metal e.g., ⁹⁹mTc, Ga-68, Lu-177, Re-186 etc., but not limited to these metals. (Schemes 14 and 15)

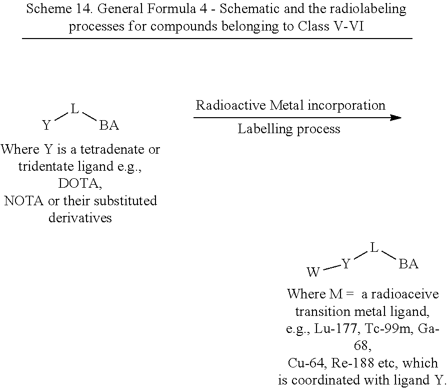

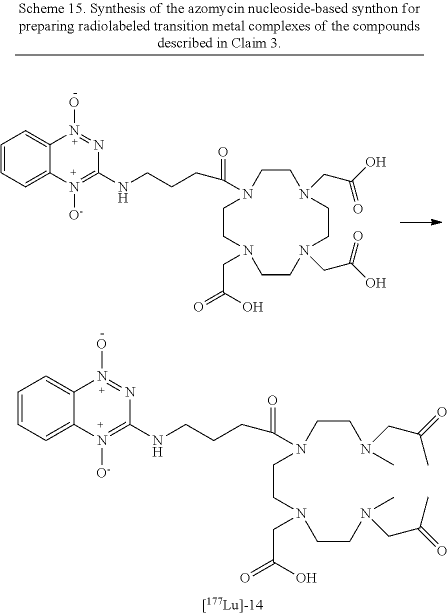

### Class VIII: General Formula 4 for Radioligand-Based Radiopharmaceuticals

Examples of this class of radiopharmaceuticals include where the molecules are chelated with an imaging or radiotherapeutic metal e.g., ^{99m}Tc, Ga-68, Lu-177, Re-186 etc., but not limited to these metals (Scheme 16).

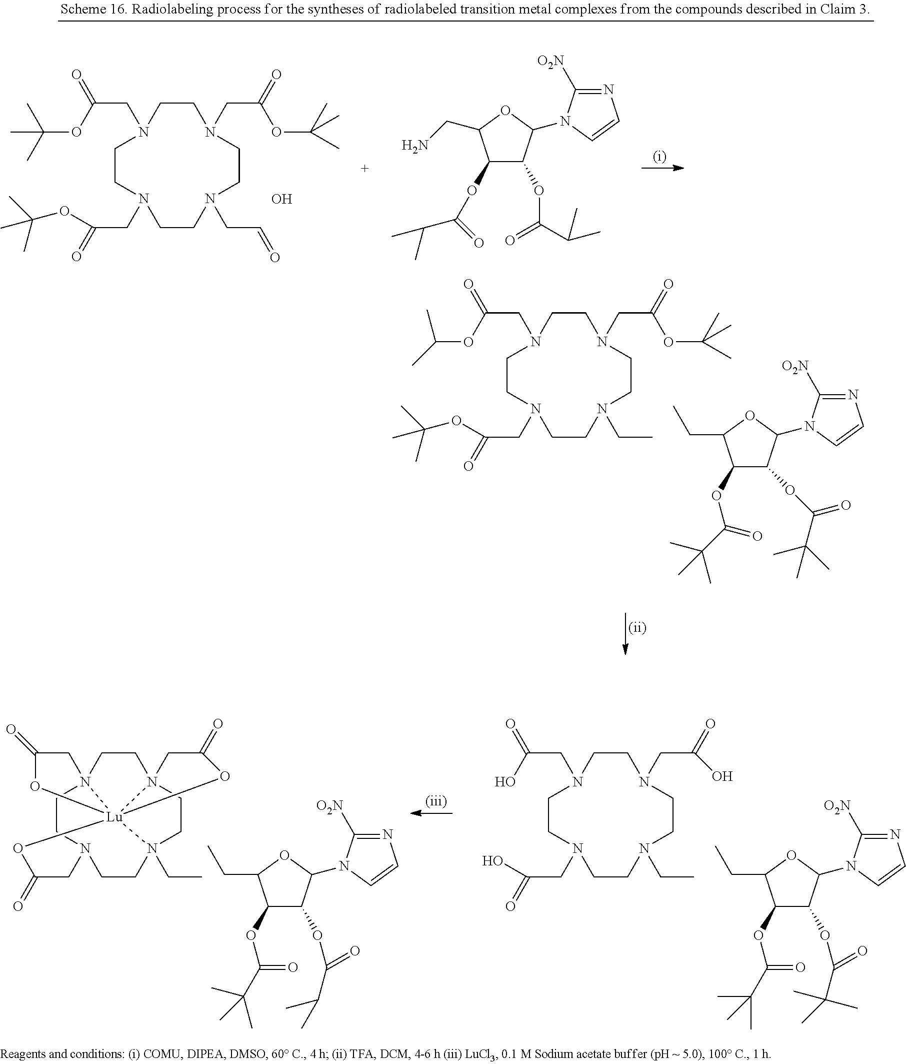

MRT, Chemosensitization Therapy, Radiosensitization Therapy, Auger Therapy, Hypoxia Imaging

(a). Molecular imaging and radiotherapy properties and effects (PET e.g., [F-18, I-124, Ga-68] and SPECT [e.g., I-131 and I-123] imaging, Chemotherapy (e.g., I-127, F-19-, and other non-radioactive compounds); Auger Therapy (I-125) and Molecular Radiotherapy [MRT; I-131, Lu-177, Re-186 but not limited to these isotopes]) of the molecules described herein, and the related processes;

(b). Theranostic uses (PET and SPECT imaging, MRT) of the molecules described above and the related processes and benefits

Biological Studies

1. In Vitro Studies

Cytotoxicity:

Exponentially-growing human cancer cells (FaDu [head & neck], U-251 [glioblastoma] and MCF-7 [breast]) cultures were trypsinized, collected and diluted in the appropriate medium to a cell concentration of 8×103 cells/mL. Cells (1.2×103-1.5×103 cells/well in 100 μL) were seeded into 96-well plates and incubated (24 h; 37° C.) under either 5% CO₂ in air, or under nitrogen. Test compounds were dissolved at the desired concentrations (1.0×10⁻³ M to 1.0×10⁻⁷ M) in growth medium, and the resulting compounds' solutions (100 μL) were added to the cell-containing wells. Hypoxic conditions under nitrogen were created by successive evacuation/refill cycles with high purity nitrogen. In controls (hypoxic and aerobic), medium (100 μL) replaced the test-compound solution. After a 72 h incubation, 3-(4,5-dimethylthiazol-2-yl)-2,5-diphenyl tetrazolium bromide (MTT, 50 μL of 1 mg/mL solution) was added to each well, and after a 4 h incubation the supernatant was removed and dimethylsulfoxide (DMSO; 150 μL) was added to each well to dissolve the formazan crystals. The well-plates were shaken for 30 min to ensure complete extraction, and then scanned at 544 nm using an ELISA reader. Survival curves for each test compounds were generated from net (test minus control) optical density data.

Cytotoxicity data for tested compounds are provided in the Figures section.

Radiosensitization:

FaDu (head & neck), U-251 (glioblastoma) and MCF-7 (breast) cancer cells (3×10⁵ cells or 5×10⁵ cells per dish in 4 mL DMEM/F12 medium per T60 glass Petri dish) were incubated (37° C., 20 h) under 5% CO₂ in air. Test drug stock solutions (10 mM in 95% ethanol) were then individually added to achieve a concentration of 10⁻⁴M or 10⁻⁵ M (depending on the test drug class), and incubation was continued for 24 h. Dishes were assigned to either the control (normoxic) or hypoxic groups. Those in the hypoxic group were de-gassed to hypoxia by 6 consecutive vacuum/nitrogen (high purity) fill cycles in a vacuum chamber. The Petri dishes (hypoxic and normoxic controls) were then incubated for 30 min on an oscillating shaker at 37° C. (60 cycles/min) and irradiated in a ⁶⁰Co γ-irradiator at various radiation doses ranging from 0 (control) to 18 Gy in either N2 (hypoxic sub-group) or air (normoxic sub-group up to 8 Gy) chambers. The cells were sequentially washed with PBS, trypsinized (500 μL), quenched with fresh medium (4.5 mL), plated in medium at densities ranging from 100 to 15,000 cells/5 mL medium (normoxic cells; 100 and 5,000 cells/5 mL medium for hypoxic cells), and then incubated (37° C.; 5% CO₂ in air). After 1 to 3 weeks of incubation, cells were stained with methylene blue or crystal violet in ethanol, clones were counted and surviving fractions calculated.

2. In Vivo Radiosensitization Therapy

In very brief, the evaluation of radiosensitization potential of IAZA using a single chemical dose (20% of the maximum tolerated dose; MTD) and a single radiation dose (10 Gy) was done in ‘bi-flank FaDu tumor-bearing NuNu mice was done. Benefits of IAZA-bestowed radiosensitization therapy effects were compared with the conventional radiotherapy alone (a single 10 Gy dose). Reduction in hypoxic content of the tumor and tumor size reduction were observed with no morbidity, when tumor-bearing mice were treated with IAZA followed by external beam radiotherapy.

The results from the described tests for selected representatives from azomycin and benzotriazene classes of drugs are provided in the figures at the end of the Claims.

1. Data from In Vitro Cytotoxicity Evaluations

a). Studies with PK-CR-IA (Mono-retinoyl IAZA; Compound 6)

FIG. 5 depicts MTT Assay for PK-CR-IA in FaDu Cells.

b) Studies with IAZA

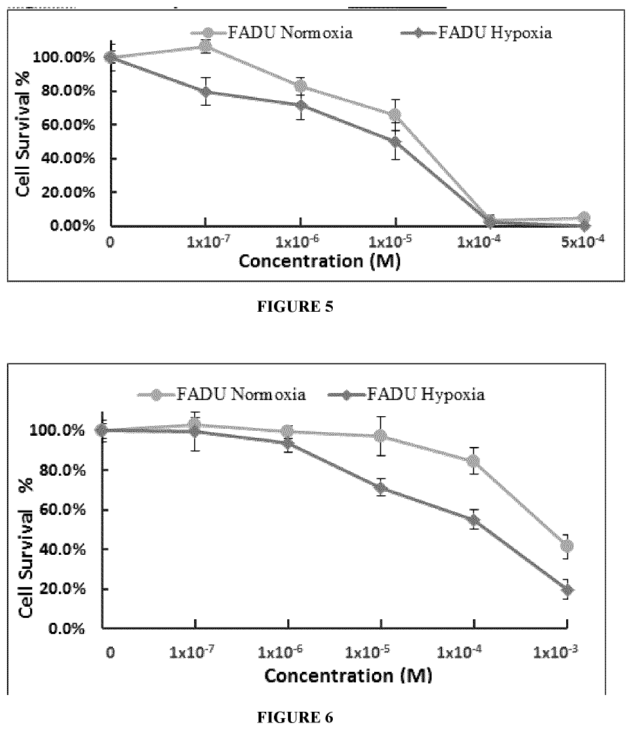

FIG. 6 depicts MTT Assay for IAZA in FaDu Cells.

c) Study with Retinoic Acid (MTS Assay)

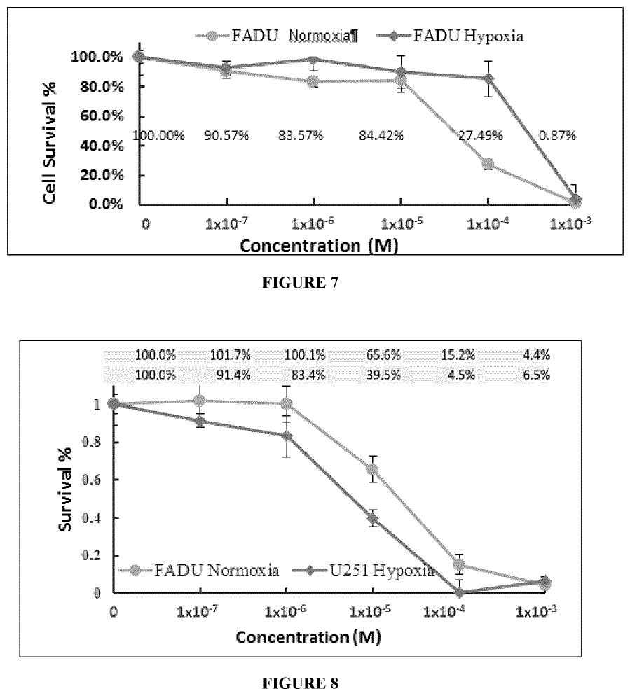

FIG. 7 depicts MTT assay for retinoic acid in FaDu cells.

d) Studies with HE-1-57-B23 (I-TPZ; Compound 9)

FIG. 8 depicts Cytotoxicity of HE-1-57-B23 in FaDu cells.

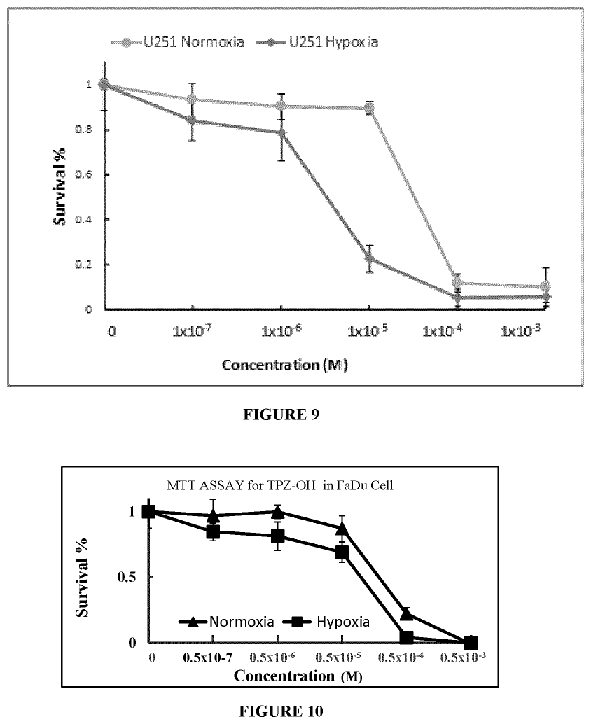

FIG. 9 depicts Cytotoxicity of HE-1-57-B23 in U251 Cells.

FIG. 10 depicts Cytotoxicity of TPZ-OH in FaDu cells.

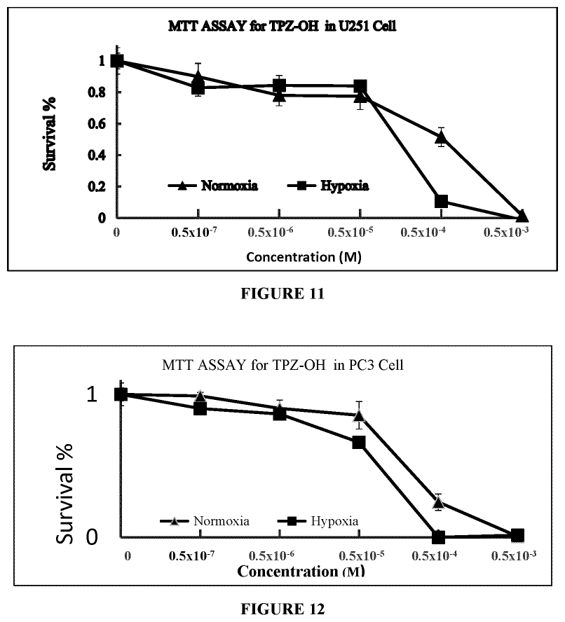

FIG. 11 depicts Cytotoxicity of TPZ-OH in U251 cells.

FIG. 12 depicts Cytotoxicity of TPZ-OH in PC3 cells

g) Studies with HE-B-104 (Compound 16)

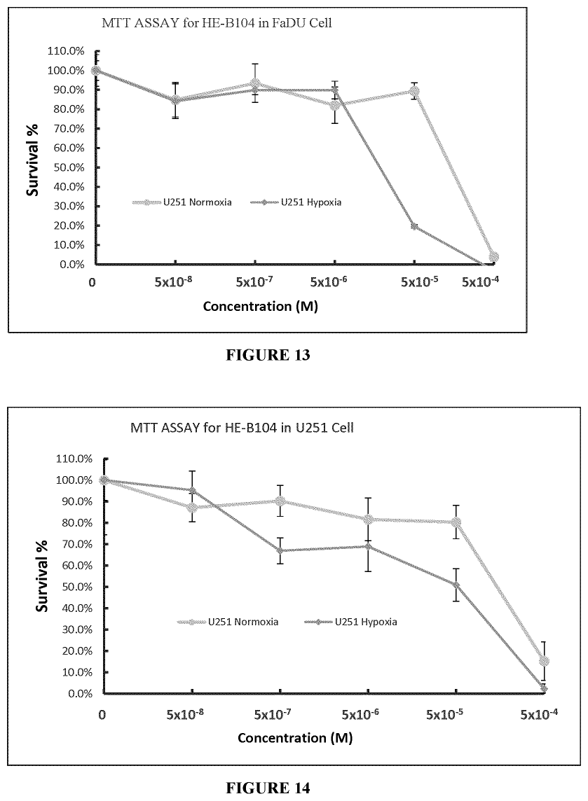

FIG. 13 depicts Cytotoxicity of HE-B-104 in FaDu cells.

FIG. 14 depicts Cytotoxicity of HE-B-104 in U251 cells.

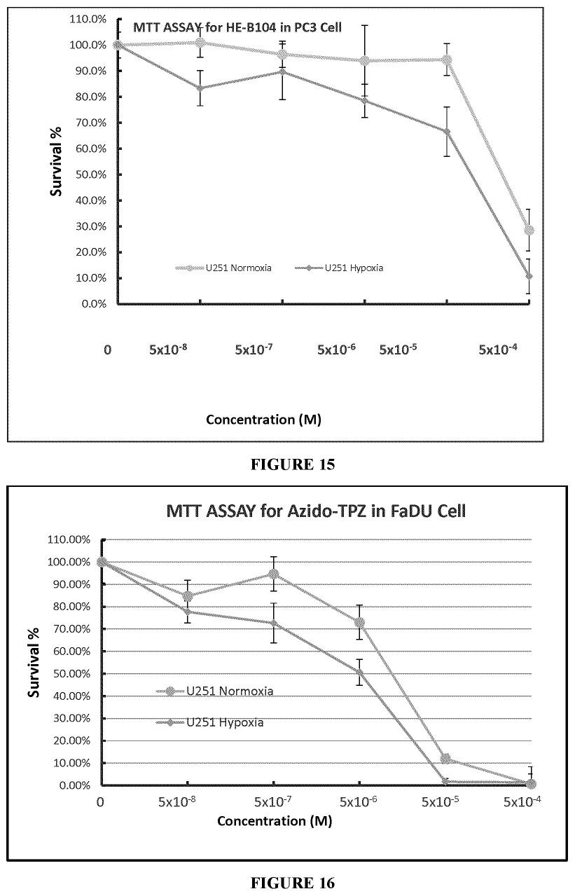

FIG. 15 depicts Cytotoxicity of HE-B-104 in PC3 cells.

g) Studies with azido-TPZ (A-TPZ; Compound 10b)

FIG. 16 depicts Cytotoxicity of Azido-TPZ in FaDu cells.

FIG. 17 depicts Cytotoxicity of Azido-TPZ in U251 cells.

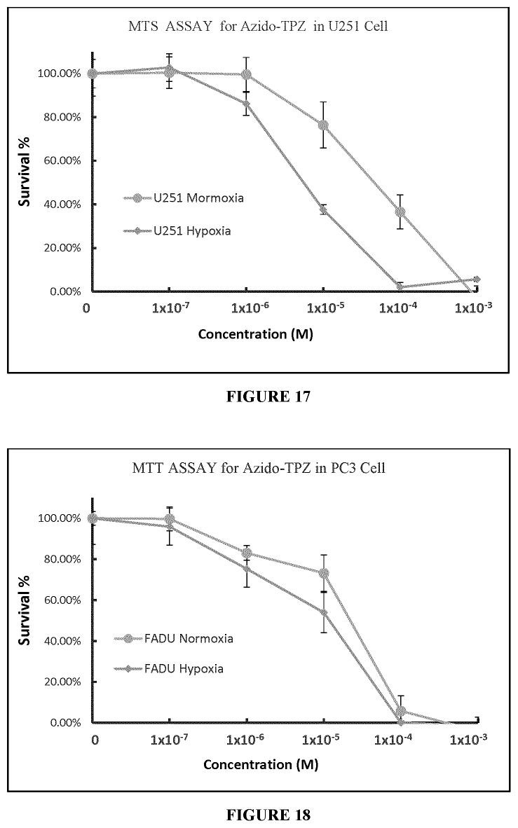

FIG. 18 depicts Cytotoxicity of Azido-TPZ in PC3 cells.

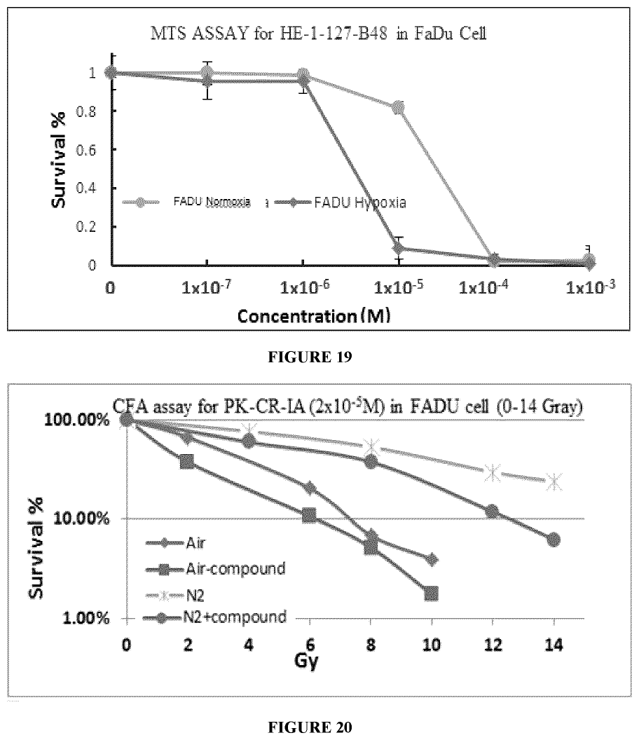

FIG. 19 depicts Cytotoxicity of HE-1-127-B48 in FaDu cells.

Data from In vitro Radiosensitization Evaluations under hypoxic conditions

a) Evaluation of PK-CR-IA (Compound 6; 2×10−5M)

FIG. 20 depicts Radiosensitization of FaDu cells by PK-CR-IA-CFA assay at 0-14 Gray.

b) Evaluation of HE-1-57-B23 at (Compound 9; 1×10−5 M)

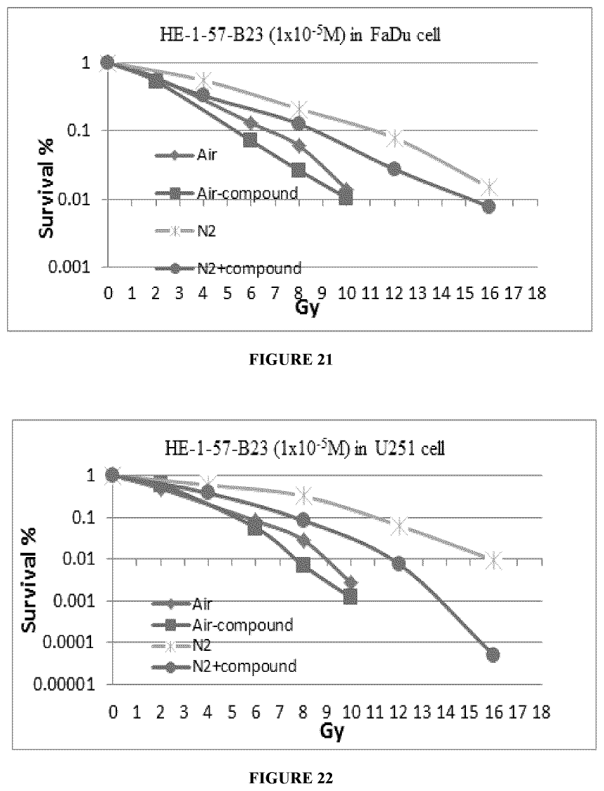

FIG. 21 depicts Radiosensitization of FaDu cells by HE-1-57-B23.

FIG. 22 depicts Radiosensitization of U-251 cells by HE-1-57-B23.

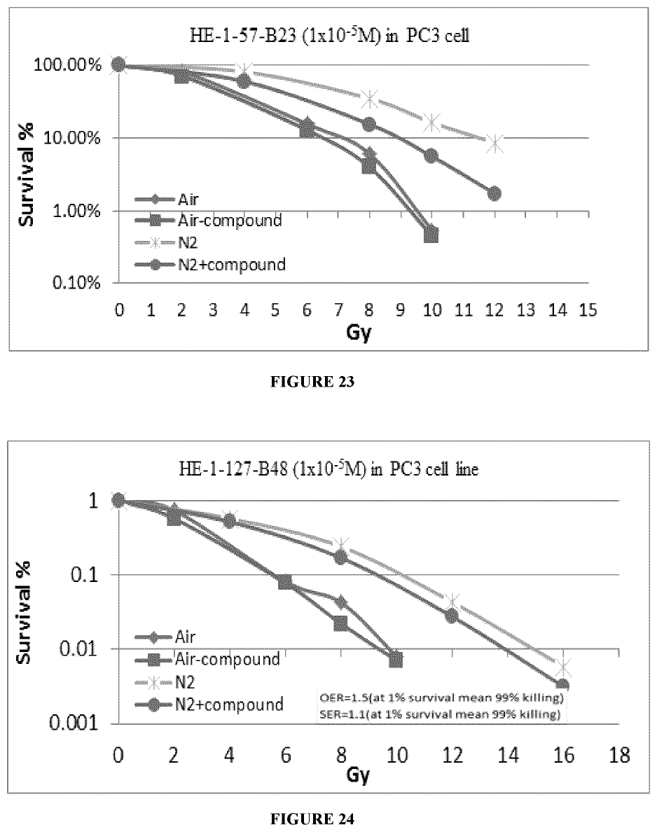

FIG. 23 depicts Radiosensitization of PC-3 cells by HE-1-57-B23.

c) Evaluation of HE-1-127-B48 (1×10−5 M)

FIG. 24 depicts Radiosensitization of PC3 cells by HE-1-127-B48.

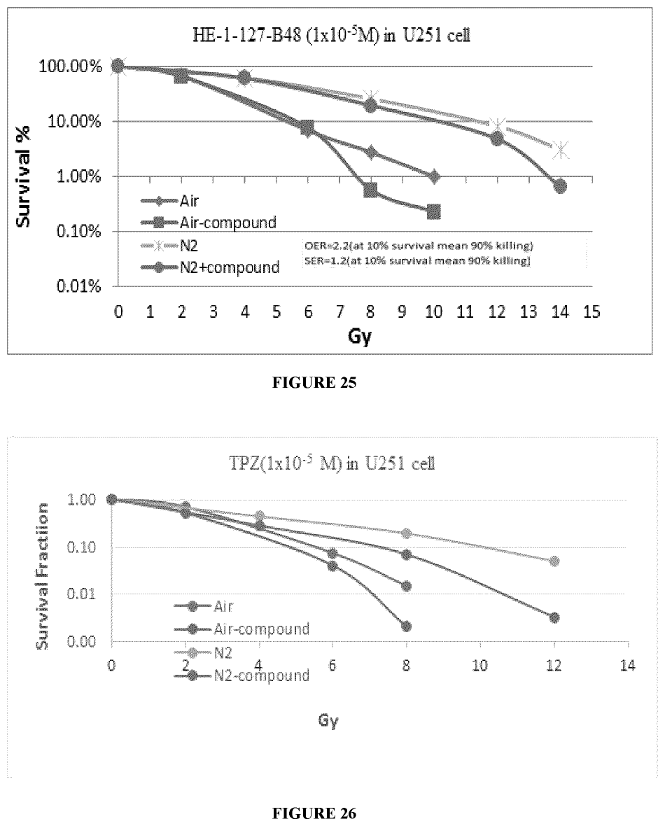

FIG. 25 depicts Radiosensitization of U251 cells by HE-1-127-B48.

d) Evaluation of Tirapazamine (TPZ; 1×10−5 M) in various cancer cells

FIG. 26 depicts Radiosensitization of U251 cells by TPZ.

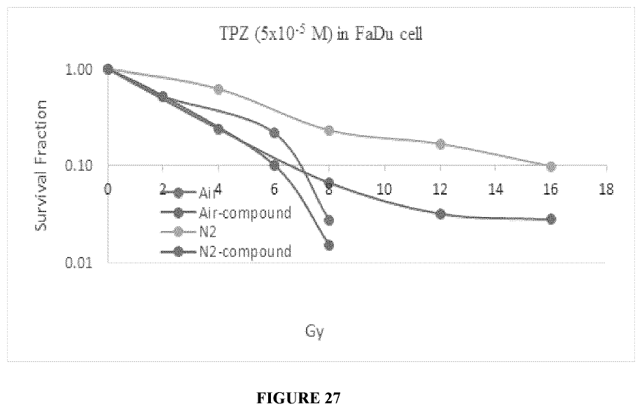

FIG. 27 depicts Radiosensitization of FaDu cells by TPZ.

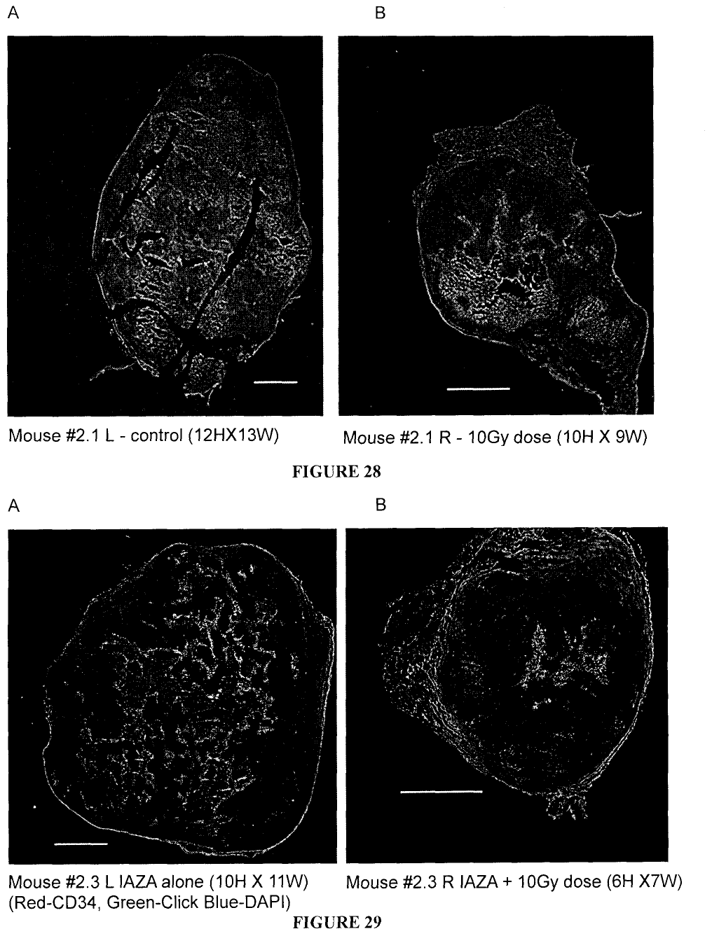

FIGS. 28 and 29 depict IN VIVO RADIOSENSITIZATION THERAPY OF FaDu TUMOR BEARING NU-NU MICE USING A SINGLE CHEMICAL DOSE OF IAZA (20% of MTD) AND 10 Gy RADIATION DOSE (Green stain indicates the hypoxic region in tumor)

FIG. 28 depict histological sections of FaDu tumors grown in mice, representing No treatment (A) and Radiation (10 Gy) alone treatment (B).

FIG. 29 depicts histological sections of FaDu tumors grown in mice, representing IAZA treatment (A) and IAZA plus Radiation (10 Gy) treatment (B).

### REFERENCES

Hall E J. Radiobiology for the radiobiologist. Pub. J.B. Lippincott Co., Philadelphia, USA. (1994).
Bennewith K L, and S Dedhar. Targeting hypoxic tumour cells to overcome metastasis. BMC Cancer. 11:504 (2011).
Padera T P, B R Stoll, J B Tooredman, D Capen, E di Tomaso, R K Jain. Pathology: cancer cells compress intratumoural vessels. Nature. 427:695-695 (2004).
Intaglietta M, R R Myers, J F Gross, H S Reinhold. Dynamics of microvascular flow in implanted mouse mammary tumors. Bibl Anat. 15:273-276 (1977).
Chaplin D J, P L Olive, R E Durand. Intermittent blood flow in a murine tumor: Radiobiological effects. Cancer Res. 47:597-601 (1987).
Minchinton A I, I F Tannock. Drug penetration in solid tumors. Nature Rev. 6:583-592 (2006).
Jain R K. Delivery of molecular and cellular medicine to solid tumors. Microcirculation. 4:3-21 (1997).
Heidin C H, K Rubin, K Pietras, AOstman. High interstitial fluid pressure—an obstacle in cancer therapy. Nature Rev Cancer. 4:806-813 (2004).
Stratton M R. P J Campbell, P A Futreal. The cancer genome. Nature. 458:719-724 (2009).
Nordsmark M, M Overgaard, J Overgaard. Pretreatment oxygenation predicts radiation response in advanced squamous cell carcinoma of the head and neck. Radiother Oncol. 41:31-9, (1996).
Bindra R S, M E Crosby, P M Glazer, A L Harris. Regulation of DNA repair in hypoxic cancer cells. Cancer Metastasis. 26:249-260, (2007).
Bencokova Z, M R Kaufmann, I M Pires, P S Lecane, E M Hammond, ATM activation and signaling under hypoxic conditions. Mol Cell Biol. 29:526-537, (2009).
Lee N Y, Q T Le. New developments in radiation therapy for head and neck cancer: intensity-modulated radiation therapy and hypoxia targeting. Semin Oncol. 35:236-50 (2008).
Chi J T, Z Wang, D S Nuyten, E H Rodriguez, M E Schaner, A Salim, Y Wang, G B Kristensen, A Helland, A L Borrasen-Dale, A Giaccia, M T Longaker, T Hastie, G P Yang, M J van der Vijver, P O Brown. Gene expression programs in response to hypoxia: cell type specificity and prognostic significance in human cancers. PloS Med. 3:e47 (2006).
Ma H I, S H Chiou, D Y Hueng, L K Tai, P I Huang, C L Kao, Y W Chen, H K Sytwu. Celecoxib and radioresistant glioblastoma-derived CD133(+) cells: improvement in radiotherapeutic effects Laboratory investigation. J Neurosurgery. 114:651-662 (2011).
Keith B, M C Simon. Hypoxia-inducible factors, Stem cells and Cancer. Cell. 129:465-472 (2007).
Seidel S, B K Garvalov, V Wirta, L von Stechow, A Schanzer, K Meletis, M Wolter, D Sommerlad, A T Henze, M Nister, G Reifenberger, J Lundenberg, J Frisen, T Acker. A hypoxic niche regulates glioblastoma stem cells through hypoxia inducible factor 2alpha. Brain. 133:983-995 (2010).
Bar E E, A Lin, V Mahairaki, W Matsui, C G Eberhart. Hypoxia increases the expression of stem-cell markers and promotes clonogenicity in glioblastoma. Neurospheres. 1491-1502 (2010).
Birner P, M Piribauer, I Fischer, B Gatterbauer, C Marosi, P F Ambros, I M Ambeos, M Bredel, G Oberhuber, K Rossler, H Budka, A L Harris, J A Hainfellner. Vascular patterns in glioblastoma influence clinical outcome and associate with variable expression of angiogenic proteins: evidence for distinct angiogenic subtypes. Brain Pathol. 13:133-143 (2003).
Blazek E R, J L Foutch, G Maki G. Daoymedulloblastoma cells that express CD133 are radioresistant relative to CD133-cells, and the CD133+ sector is enlarged by hypoxia. Int J Radiat Oncol Biol Phys. 67:1-5 (2007).
Bristow R G, R P Hill. Hypoxia and metabolism: Hypoxia, DNA repair and genetic instability. Nat Rev Cancer. 8:180-92 (2008).
Clement V, V Dutoit, D Marino, P Y Dietrich, I Radovanovic. Limits of CD133 as a marker of glioma self-renewing cells. Int J Cancer. 125:244-248 (2009).
Brown J M. The hypoxic cell: a target for selective cancer therapy-eighteenth Bruce F. Cain memorial award lecture. Cancer Res. 59:5863-5870 (1999).

Kits

Method of the invention are conveniently practiced by providing the compounds and/or compositions used in such method in the form of a kit. Such kit preferably contains the compound(s) and/or composition(s).

As used herein, the term “instructions for administering said compound to a subject,” and grammatical equivalents thereof, includes instructions for using the compositions contained in a kit for the treatment of conditions characterized by viral infection (e.g., providing dosing, route of administration, decision trees for treating physicians for correlating patient-specific characteristics with therapeutic courses of action). The compounds of the present invention (e.g. as shown in structures above and elsewhere presented herein) can be packaged into a kit, which may include instructions for administering the compounds to a subject.

It should be understood that the examples herein are for illustrative purposes only. Therefore, they should not limit the scope of this invention in anyway.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

What is claimed is:

1. A compound of formula (I), or any prodrug, pharmaceutically acceptable salt, metabolite, polymorph, solvate, hydrate, stereoisomer, radioisotope or tautomer thereof 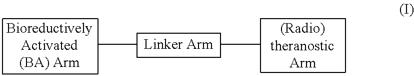
wherein BA comprises one or more of 2/4/5-substituted nitroimidazoles, substituted benzotriazene-1,4-dioxides, substituted 1,2,3/1,2,4-triazoles, substituted 1,4-benzoquinones, of combination of two homo- or hetero BA moieties,
wherein Linker Arm is —C₁₋₁₆ alkane, alkene, alkyne, alicyclic, aromatic with or without hetero atoms as in ethers, amines, esters, acids, amides; 5 and 6 membered rings with the substitutions as described above, both monosaccharides and diasaccharides,
wherein the (Raio)theranostic Arm comprises ^{18/19}F, ^{123/124/125/127/131}I, Lu-177, Ga-68, ^{99m}TC, etc.

2. A compound of formula (II), or any prodrug, pharmaceutically acceptable salt, metabolite, polymorph, solvate, hydrate, stereoisomer, radioisotope or tautomer thereof, 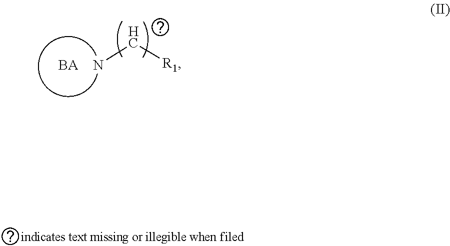
wherein 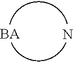
(BA) is a bioreductively-activated molecule, for example, 2/4/5-nitroimidazoles (such as in F-MISO), or substituted with cyclic moieties, or sugar substituted moieties (both pentoses as in FAZA [substituted or unsubstituted] and IAZA [substituted or unsubstituted], and hexoses, disaccharides and trisaccharides in all configurations; for example, as in glucoses, galactoses, fructoses, other substituted moieties nitroimidazoles, benzotriazene-1,4-dioxides e.g. tirapazamine, and analogs thereof, substituted 1,2,4-triazoles, substituted tetrahydroisoquinolines, substitutes benzoquinones, e.g. AQ4N;
wherein R₁ is unsubstituted, or substituted molecule with one or more —OH groups, wherein the one or more —OH group is substituted with an alkyl, aralkyl ether, ester, amine or a thiol, and the remaining free —OH group is replaced by a radiohalogen, H, halogen, azide, amine-substituted/unsubstituted, —OH, substituted —OH, —OSO₂R₃;
wherein R₃ is alkyl sulfonyl (such as methanesulfonyl, or arylsulfonyl e.g. tosyl, nosyl, trifly)-substituted alkan/alkene/alkyne/alkoxy/alkoxyalkenyl and alkoxyalkynyl chains;
wherein n is C₁-C₂₂.

3. The compound of claim 2, wherein the sugar containing bioreductively activated molecule is substituted with an ether or ester moiety at 2′ and/or 3′ positions, and a halogen/pseudohalogen (F/I/OTosyl/ONosyl/OTriflyl/OMesyl) substituted at 2′- or 3′ or 5′ —OH of a sugar with or without a linker.

4. The compound of claim 2 or 3, wherein said Acyclic or cyclic substituents linked to the BA moieties are further substituted with R1, where R1=alkane/alkene/alkyne/alkoxy/alkoxyalkyl/alkoxyalkenyl and alkoxyalkynyl chains (C1-C22), where R2═H, halogens, Azide, —OH, substituted —OH, —OSO2R3 (R3=alkyl sulfonyl e.g., methanesulfonyl, or arylsulfonyl e.g., tosyl, nosyl, triflyl).

5. The compound of any one of claims 2 to 4, wherein when said bioreductively activated molecule is an azomycin-based compound, such as retinoyl IAZA [Ret-IAZA], retinoyl FAZA [Ret-FAZA], but are not limited to sugar conjugated family; in benzotriazene-1,4-dioxide based molecules include tirapazamine (TPZ)-based compounds, for example (C2/C4/C6 gluc substituted-TPZ), and all related precursors to synthesize the corresponding halogenated (F, Cl, Br, I, At) derivatives.

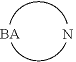

6. The compound of any one of claims 1 to 4, wherein said is

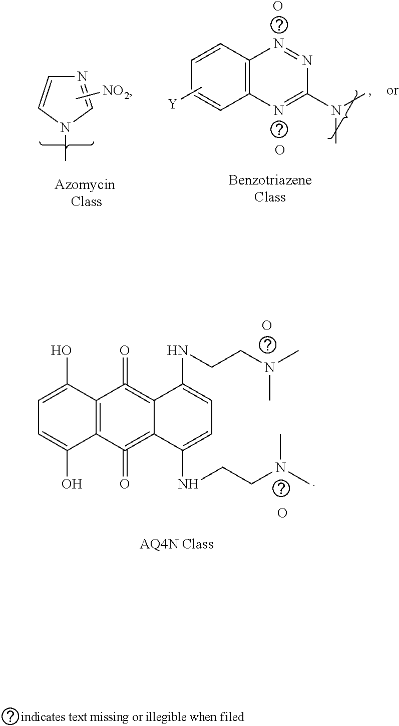

7. The compound of claim 6, wherein said benzotriazene class is 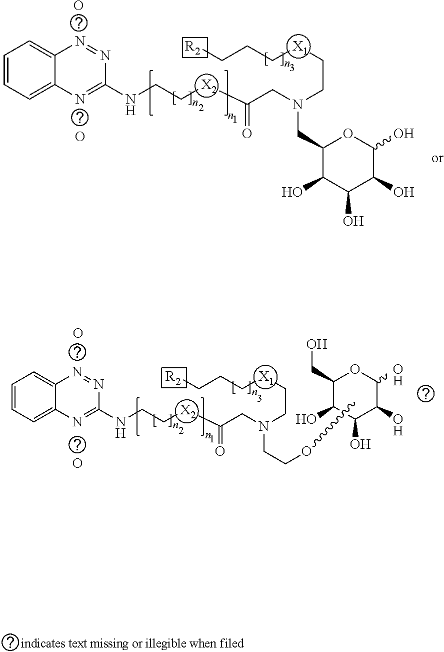
wherein R2 is I, F, Br, Cl, At, N3;
wherein X1 is C, N, O, S;
wherein X2 is C, N, O, S
wherein n1 is 1-22;
wherein n2 is 1-22;
wherein n3 is 1-22.

8. A compound of formula (II), or any prodrug, pharmaceutically acceptable salt, metabolite, polymorph, solvate, hydrate, stereoisomer, radioisotope or tautomer thereof,
Y-L-BA (II)
wherein BA is a bioreductively-activated molecule, for example, 2/4/5-nitroimidazoles (such as in F-MISO), or substituted with cyclic moieties, or sugar substituted moieties (both pentoses as in FAZA [substituted or unsubstituted] and IAZA [substituted or unsubstituted], and hexoses, disaccharides and trisaccharides in all configurations; for example, as in glucoses, galactoses, fructoses, other substituted moieties nitroimidazoles, benzotriazene-1,4-dioxides e.g. tirapazamine, and analogs thereof, substituted 1,2,4-triazoles, substituted tetrahydroisoquinolines, substitutes benzoquinones, e.g. AQ4N;
wherein L is a linker, such as cyclic or acyclic moiety with up to C8 chain, which can be further substituted by alkane/alkene/alkyne/alkoxy/alkoxyalkyl/alkoxyalkenyl or alkoxyalkynyl chains (C1-C22) containing H, halogen, azide, —OH, substituted —OH, —OSO₂R₃(R₃ is alkyl sulfonyl e.g., methanesulfonyl or arylsulfonyl e.g. tosyl, nosyl, triflyl), for example C1-α/β-substituted arabinofuranoses/pentoses/hexoses (e.g., glucose, disaccharide etc.) where the other —OH groups except one in the sugar ring are either unsubstituted, or substituted with alkyl aralkyl ethers, esters, amines or thiols; remaining free —OH group is replaced by radio halogen,
wherein Y is a ligand (e.g., tetradentate ligand for example DOTA or NOTA or PnAO.

9. A radio labeled compound comprising a compound of any one of claims 1 to 8, wherein said radio label is a radioisotope, a radiohalogen, F-18, I-123/124/125/131, F-18 labelled dipivaloyl 5′-¹⁸FAZA and I-123/124/125/131-labelled diretinoyl-^{123/124/125/131}IAZA, radiolabeled ret-IAZA or retinoyl FAZA, for both α- and β-conformers.

10. A pharmaceutical comprising a compound of any one of claims 1 to 9, or a radio labeled compound of claim 8, and one or more inert carriers and/or diluents.

11. Use of a compound of any one of claims 1 to 8, a radio labeled compound of claim 9, or a pharmaceutical composition of claim 10, as a diagnostic agent in a subject.

12. Use of a compound of any one of claims 1 to 8, a radio labeled compound of claim 9, or a pharmaceutical composition of claim 10, as a therapeutic agent in a subject.

13. Use of a compound of any one of claims 1 to 8, a radio labeled compound of claim 9, or a pharmaceutical composition of claim 10, as a diagnostic and therapeutic agent in a subject.

14. Use of a compound of any one of claims 1 to 8, a radio labeled compound of claim 9, or a pharmaceutical composition of claim 10, as an imaging agent in a subject.

15. Use of a compound of any one of claims 1 to 8, a radio labeled compound of claim 9, or a pharmaceutical composition of claim 10, as a radiosensitization agent in a subject.

16. Use of a compound of any one of claims 1 to 8, a radio labeled compound of claim 9, or a pharmaceutical composition of claim 10, as a chemosensitization agent in a subject.

17. Use of a compound of any one of claims 1 to 8, a radio labeled compound of claim 9, or a pharmaceutical composition of claim 10 in the treatment of a hypoxia tumours and/or cancers, diabetes, inflammatory arthritis, anaerobic bacterial infection, stroke, brain trauma or transplant rejection.

18. The use of any one of claims 11 to 17, wherein said subject is a human.
